(19) United States
(12) Patent Application Publication
(10) Pub. No.: US 2011/0098188 A1
(43) Pub. Date: Apr. 28, 2011

(54) BLOOD BIOMARKERS FOR PSYCHOSIS

(75) Inventors: Alexander B. Niculescu, Indianapolis, IN (US); Daniel R. Salomon, San Diego, CA (US)

(73) Assignees: THE SCRIPPS RESEARCH INSTITUTE, La Jolla, CA (US); INDIANA UNIVERSITY RESEARCH AND TECHNOLOGY CORPORATION, Indianapolis, IN (US)

(21) Appl. No.: 12/599,763

(22) PCT Filed: May 13, 2008
(86) PCT No.: PCT/US08/63539

§ 371 Date: Dec. 22, 2010

Related U.S. Application Data
(60) Provisional application No. 60/917,784, filed on May 14, 2007.

(51) Int. Cl. C40B 30/04; C12Q 1/68; G01N 33/53; C40B 40/04; C40B 40/10

(52) U.S. Cl. 506/9; 435/6; 435/7.92; 506/15; 506/18

(57) ABSTRACT

A plurality of biomarkers determine the diagnosis of psychosis based on the expression levels in a sample such as blood. Subsets of biomarkers predict the diagnosis of delusion or hallucination. The biomarkers are identified using a convergent functional genomics approach based on animal and human data. Methods and compositions for clinical diagnosis of psychosis are provided.

### CROSS REFERENCE TO RELATED APPLICATIONS

This application claims priority to U.S. provisional application Ser. No. 60/917,784, filed May 14, 2007, the disclosure of which is hereby incorporated by reference in its entirety.

Part of the work during the development of this invention was made with government support from the National Institutes of Health under grant NIMH R01 MH071912-01. The U.S. Government has certain rights in the invention.

### BACKGROUND

Research into the biological basis of psychotic disorders (such as schizophrenia and schizoaffective disorder) has been primarily focused in human and animal studies mostly independently. The two avenues of research have complementary strengths and weaknesses. In human genetic studies, for example, in samples of patients with psychotic disorders and their family members, positional cloning methods such as linkage analysis, linkage-disequilibrium mapping, and candidate-gene association analysis are narrowing the search for the chromosomal regions harboring risk genes for the illness and, in some cases, identifying plausible candidate genes and polymorphisms that will require further validation. Human postmortem brain gene expression studies have also been employed as a way of trying to identify candidate genes for psychotic disorders. In general, human studies suffer from issues of sensitivity—the signal is often difficult to detect due to the noise generated by the genetic heterogeneity of individuals and the effects of diverse environmental exposures on gene expression and phenotypic penetrance.

In animal studies, carried out in isogenic strains with controlled environmental exposure, the identification of putative neurobiological substrates of psychotic disorders is typically accomplished by modeling human psychotic disorders through pharmacological or genetic manipulations. Animal model studies suffer from issues of specificity—questions regarding direct relevance to the human disorder modeled. Each independent line of investigation (i.e., human and animal studies) is contributing to the incremental gains in knowledge of psychotic disorders etiology witnessed in the last decade.

However, a lack of integration between these two lines of investigations hinders scientific understanding and slows the pace of discovery. Psychiatric phenotypes, as currently defined, are primarily the result of clinical consensus criteria rather than empirical determination. The present disclosure provides methods and compositions that empirically determine disease states for diagnosis and treatment.

Objective biomarkers of illness and treatment response would make a significant difference in the ability to diagnose and treat patients with psychotic disorders, eliminating subjectivity and reliance of patient's self-report of symptoms. Blood gene expression profiling has emerged as a particularly interesting area of research in the search for peripheral biomarkers. Most of the studies to date have focused on human lymphocytes gene expression profiling, comparison between illness groups and normal controls. They suffer from one of both of the following limitations: 1) the sample size used is often small. Given the genetic heterogeneity in human samples and the effects of illness state and environmental history, including medications and drugs, on gene expression, it may not be reliable to extract bona fide findings. 2) Use of lymphoblastoid cell lines—passaged lymphoblastoid cell lines provide a self-renewable source of material, and are purported to avoid the effects of environmental exposure of cells from fresh blood. Fresh blood, however, with phenotypic state information gathered at time of harvesting, may be more informative than immortalized lymphocytes, and may avoid some of the caveats of Epstein-Barr virus (EBV) immortalization and cell culture passaging.

The current state of the understanding of the genetic and neurobiological basis for psychotic disorders in general and of peripheral molecular biomarkers of the illness in particular, is still inadequate. Almost all of the fundamental genetic, environmental, and biological elements needed to delineate the etiology and pathophysiology of psychotic disorders are yet to be completely identified, understood and validated. One of the rate-limiting steps has been the lack of concerted integration across disciplines and methodologies. The use of a multidisciplinary, integrative research framework as in the present disclosure provided herein, should lead to a reduction in the historically high rate of inferential errors committed in studies of complex diseases like psychotic disorders.

Identification and validation of peripheral biomarkers for psychotic disorders have proven arduous, despite recent large-scale efforts. Human genomic studies are susceptible to the issue of being underpowered, due to genetic heterogeneity, the effect of variable environmental exposure on gene expression, and difficulty of accrual of large samples. Animal model gene expression studies, in a genetically homogeneous and experimentally tractable setting, can avoid artifacts and provide sensitivity of detection. Subsequent comparisons of the animal datasets with human genetic and genomic datasets can ensure cross-validatory power and specificity.

Convergent functional genomics (CFG), is an approach that translationally cross-matches animal model gene expression data with human genetic linkage data and human tissue data (blood, postmortem brain), as a Bayesian strategy of cross validating findings and identifying candidate genes, pathways and mechanisms for neuropsychiatric disorders. Predictive biomarkers for psychosis are desired for clinical diagnosis and treatment purposes. The present disclosure provides several biomarkers that are predictive of psychotic disorders in clinical settings.

No objective clinical laboratory blood tests for psychosis is available. The current reliance on patient self-report of symptom severity and on the clinicians' impression alone are rate limiting steps in effective treatment, and in new drug development. Blood biomarkers for psychosis state provide useful tools for diagnosis and therapy.

### SUMMARY

Methods and compositions to clinically diagnose psychotic disorders using a panel of biomarkers are disclosed. A panel of biomarkers may include 1 to about 100 or more biomarkers. The panel of biomarkers includes one or more biomarkers for psychosis. Blood is a suitable sample for measuring the levels or presence of one or more of the biomarkers provided herein.

In an aspect, psychotic symptoms measured in a quantitative fashion at time of blood draw in human subjects focus on all or nothing phenomena (genes turned on and off in low symptom states vs. high symptom states). Some of the biomarkers have cross-matched animal and human data, using a convergent functional genomics approach and from blood datasets from animal models.

Prioritized list of high probability blood biomarkers, provided herein, for psychotic disorders using cross-matching of animal and human data, provide a unique predictive power of the biomarkers, which have been experimentally tested.

Integration of human and animal model data, as a way of reducing the false-positives inherent in each approach and helping identify true biomarker molecules were adopted. Whole-genome gene expression differences were measured in fresh blood samples from patients with schizophrenia and related disorders that had no symptoms of hallucinations or delusions vs. those that had high symptoms at the time of the blood draw, and separately, changes in gene expression in the brain and blood of a mouse pharmacogenomic model. Human blood gene expression data was integrated with animal model gene expression data, human genetic linkage/association data, and human postmortem data, an approach called Convergent Functional Genomics, as a Bayesian strategy for cross-validating and prioritizing findings.

Candidate biomarker genes for hallucinations, include four genes decreased in expression in high hallucinations states (Rhobtb3, Aldh111, Mpp3, Fn1), and two genes increased in high hallucinations states (Arhgef9, S100a6). Five of these genes have evidence of differential expression in human postmortem brains from schizophrenia patients. A predictive score developed based on a panel of 10 top candidate biomarkers (5 for no hallucinations, 5 for high hallucinations) shows sensitivity and specificity for high hallucinations and no hallucinations states, in two independent cohorts.

Candidate biomarker genes for delusions include eight genes decreased in expression in high delusions states (Drd2, ApoE, Nab1, Idh1, Scamp1, Ncoa2, Aldh111, Gpm6b), and eight genes increased in high delusions states (Nrg1, Egr1, Dctn1, Nmt1, Pllp, Pvalb, Nmt1, Pctk1). Fourteen of these genes have evidence of differential expression in human postmortem brains from schizophrenia patients. A predictive score developed based on a panel of 10 top candidate biomarkers (5 for no delusions, 5 for high delusions) shows sensitivity and specificity for high delusions and no delusions states. Blood biomarkers offer an unexpectedly informative window into brain functioning and psychotic diseases states.

A method of diagnosing psychosis in an individual, the method includes:
(a) determining the expression of a plurality of biomarkers for delusion or hallucination in a sample from the individual, the plurality of biomarkers selected from the group of biomarkers listed in Table 5A, Table 5B, Table 6A, and Table 6B; and
(b) diagnosing the presence or absence of psychosis in the individual based on the expression of the plurality of biomarkers.

A plurality of biomarkers include a subset of about 10 biomarkers for delusions designated as Drd2, ApoE, Scamp1, Idh1, Nab1, Nrg1, Egr1, Dctn1, Pllp, and Pvalb or a subset of about 10 biomarkers for hallucinations designated as Rhobtb3, Aldh111, Mpp3, Fn1, Spp1, Arhgef9, S100a6, Adamts5, Pdap1, and Plxnd1.

A suitable sample is blood. The level of the biomarker can also be determined in a tissue biopsy sample of the individual. The level of the biomarker is determined by a method selected from the group that includes analyzing the expression level of RNA transcripts, analyzing the level of protein, and analyzing the level of peptides or fragments thereof. Suitable analytical techniques include microarray gene expression analysis, polymerase chain reaction (PCR), real-time PCR, quantitative PCR, immunohistochemistry, enzyme-linked immunosorbent assays (ELISA), and antibody arrays. The level of the plurality of biomarkers is performed by an analysis for the presence or absence of the biomarkers.

A method of predicting the likelihood of a successful treatment for psychosis in a patient includes:
(a) determining the expression level of at least 10 biomarkers for delusion and 10 biomarkers for hallucination, wherein the biomarkers comprise a subset of biomarkers designated as Drd2, ApoE, Scamp1, Idh1, Nab1, Nrg1, Egr1, Dctn1, Pllp, and Pvalb for delusion and Rhobtb3, Aldh111, Mpp3, Fn1, Spp1, Arhgef9, S100a6, Adamts5, Pdap1, and Plxnd1 are present for hallucination; and
(b) predicting the likelihood of successful treatment for psychosis by determining whether the sample from the patient expresses biomarkers for delusion or hallucination.

A method of treating a patient suspected of suffering psychosis includes:
(a) diagnosing whether the patient suffers from psychosis by determining the expression level of one or more of the biomarkers listed in Tables 5A, 5B, 6A, 6B in a sample obtained from the patient;
(b) selecting a treatment for psychosis based on the determination whether the patient suffers from delusion or hallucination; and
(c) administering to the patient a therapeutic agent capable of treating psychosis.

A treatment plan may include a personalized plan for the patient. A diagnostic microarray for psychosis includes a plurality of nucleic acid molecules representing genes selected from the group of genes listed in Tables 5A-5B and 6A-6B. The diagnostic microarray may consist essentially of biomarkers listed in Table 3A-3B.

A diagnostic microarray may consist essentially of biomarkers designated as Drd2, ApoE, Scamp1, Idh1, Nab1, Nrg1, Egr1, Dctn1, Pllp, and Pvalb for delusion and Rhobtb3, Aldh111, Mpp3, Fn1, Spp1, Arhgef9, S100a6, Adamts5, Pdap1, and Plxnd1 for hallucination.

A diagnostic antibody array includes a plurality of antibodies that recognize one or more epitopes corresponding to the protein products of the biomarkers designated as Drd2, ApoE, Scamp1, Idh1, Nab1, Nrg1, Egr1, Dctn1, Pllp, and Pvalb for delusion and Rhobtb3, Aldh111, Mpp3, Fn1, Spp1, Arhgef9, S100a6, Adamts5, Pdap1, and Plxnd1 for hallucination. The diagnostic antibody array may detect the protein levels of the biomarkers from a blood sample.

A kit for diagnosing psychosis includes a component selected from the group of (i) oligonucleotides for amplification of one or more genes listed in Tables 5A-5B and 6A-6B (ii) immunohistochemical agents capable of identifying the protein products of one or more biomarkers listed in Tables 5A-5B and 6A-6B (iii) the microarray of disclosed herein, and (iv) a biomarker expression index representing the genes listed in Tables 5A-5B and 6A-6B for correlation.

### BRIEF DESCRIPTION OF THE DRAWINGS

FIG. 1 shows Prioritization (A) and Conceptualization (B) of results: A.) Convergent Functional Genomics (CFG) approach for candidate biomarker prioritization. Scoring of independent lines of evidence (maximum score=9 points); B.) Conceptualization of Blood Candidate Biomarker Genes: I—Genes for the illness, whose expression is modulated by medications and by interactions with the environment (stress, drugs, al.); II—Genes for the illness, whose expression is modulated by medications; IIIa—Genes whose expression is modulated by medications and by interactions with the environment (stress, drugs, al.); IIIb—Genes for the illness, whose expression is by interactions with the environment (stress, drugs, al.); IVa—Genes whose expression is modulated by medications.

FIG. 2A illustrates some of the candidate biomarker genes for delusions (P1). Both Human Postmortem Brain and Human Blood significance was used. Both Mouse brain and Mouse Blood (italicized); Co-directional in brain-blood*; Convergence with Human Genetic Linkage to Schizophrenia; the high delusions score are not circled; circled genes—associated with low delusions score; 2B illustrates some of the candidate biomarker genes for hallucinations (P3). Both Human Postmortem Brain and Human Blood; Both Mouse brain and Mouse Blood (italicized); Co-directional in brain-blood*; Convergence with Human Genetic Linkage to Schizophrenia; high hallucinations score (non-circled); associated with low hallucinations score (circled).

FIG. 3 shows comparison of BioM-10 Hallucinations Prediction Score and actual hallucinations scores in the primary cohort of psychosis subjects (A) (n=31) and secondary psychosis cohort (B) (n=14). For hallucinations scores: blue—no hallucinations. red—high hallucinations, white—intermediate hallucinations. Hallucinations scores are based on PANSS scale administered at time of blood draw. For biomarkers: A (blue)—called Absent by MASS analysis. P (red)—called Present by MASS analysis. M (yellow)—called Marginally Present by MASS analysis. A is scored as 0, M as 0.5 and P as 1. BioM Hallucinations Prediction Score is based on the ratio of the sum of the scores for high mood biomarkers and sum of scores for low mood biomarkers, multiplied by 100. A cutoff score of 100 and above was used for high delusions. inf—infinity-denominator is 0. ND—not determined.

FIG. 4 shows comparison of BioM-10 Delusions Prediction Score and actual delusions scores in the primary cohort of psychosis subjects (A) (n=31) and secondary psychosis cohort (B) (n=14). For delusions scores: blue—no delusions. red—high delusions, white—intermediate delusions. Delusions scores are based on PANSS scale administered at time of blood draw. For biomarkers: A (blue)—called Absent by MASS analysis. P (red)—called Present by MASS analysis. M (yellow)—called Marginally Present by MASS analysis. A is scored as 0, M as 0.5 and P as 1. BioM Delusions Prediction Score is based on the ratio of the sum of the scores for high mood biomarkers and sum of scores for low mood biomarkers, multiplied by 100. A cutoff score of 100 and above was used for high delusions. inf—infinity-denominator is 0.

### DETAILED DESCRIPTION

In an aspect, the biomarkers disclosed herein are (i) derived from fresh blood, not immortalized cell lines; (ii) capable of providing quantitative psychosis information obtained at the time of the blood draw; (iii) were derived from comparisons of extremes of low delusion/high delusion and low/high hallucination in patients, as opposed to patients vs. normal controls (where the differences could be due to a lot of other environmental factors, medication (side) effects vs. no medications; (iv) based upon a smaller sample size and yet robust in their predictive power; (v) scored based on an all or nothing (Absent/Present) call for gene expression changes, not incremental changes in expression—statistically more robust and avoids false positives; (vi) based on integration of multiple independent lines of evidence that permits extraction of signal from noise (large lists of genes), and prioritization of top candidates; and (vii) used to form the basis of prediction score algorithm based.

Integration of animal model and human data were used as a way of reducing the false-positives inherent in each approach and helping identify true biomarker molecules. Gene expression differences were measured in fresh blood samples from patients with psychotic disorders (delusions/hallucinations) at the time of the blood draw. Separately, changes in gene expression were measured in the brains and bloods of a mouse pharmacogenomic models. Human blood gene expression data was integrated with animal model gene expression data, human genetic linkage/association data, and human postmortem data for cross-validating and prioritizing findings.

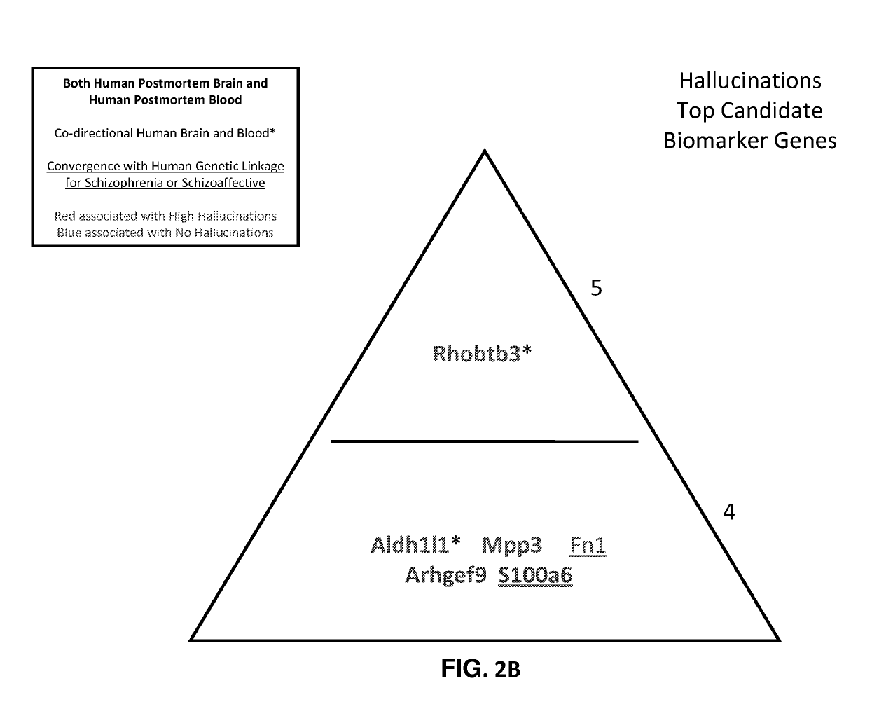

Gene expression changes in specific brain regions and blood from a pharmacogenomic animal model developed in the group were used as cross-validators to help with the identification of potential human blood biomarkers. The pharmacogenomic mouse model of relevance to psychosis consists of treatments with an agonist of the illness/psychosis-mimicking drug (phencyclidine, PCP) and an antagonist of the illness/psychosis-treating drug (clozapine). The pharmacogenomic approach is a tool for tagging genes that may have pathophysiological relevance. As an added advantage, some of these genes may be involved in potential medication effects present in human blood data (FIG. 2).

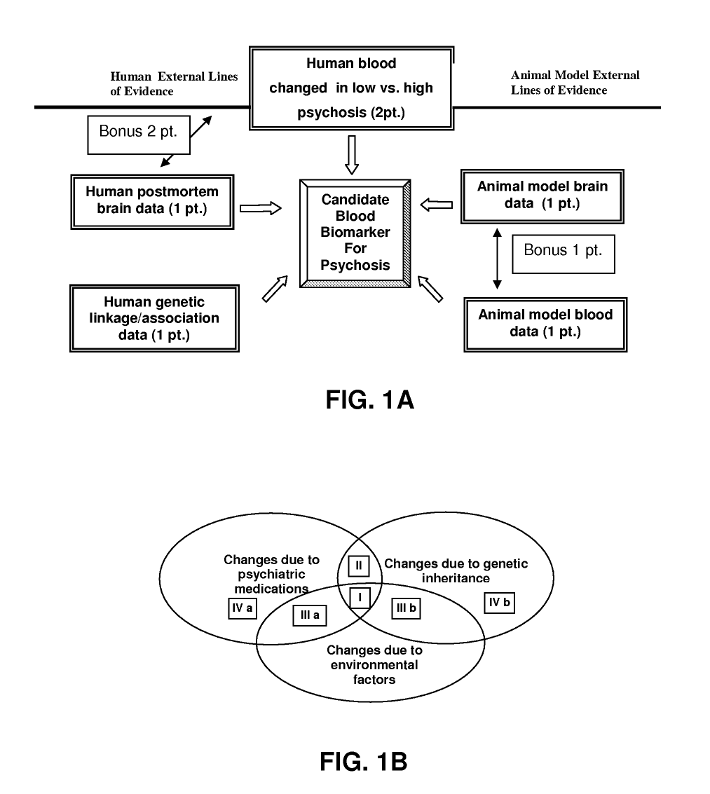

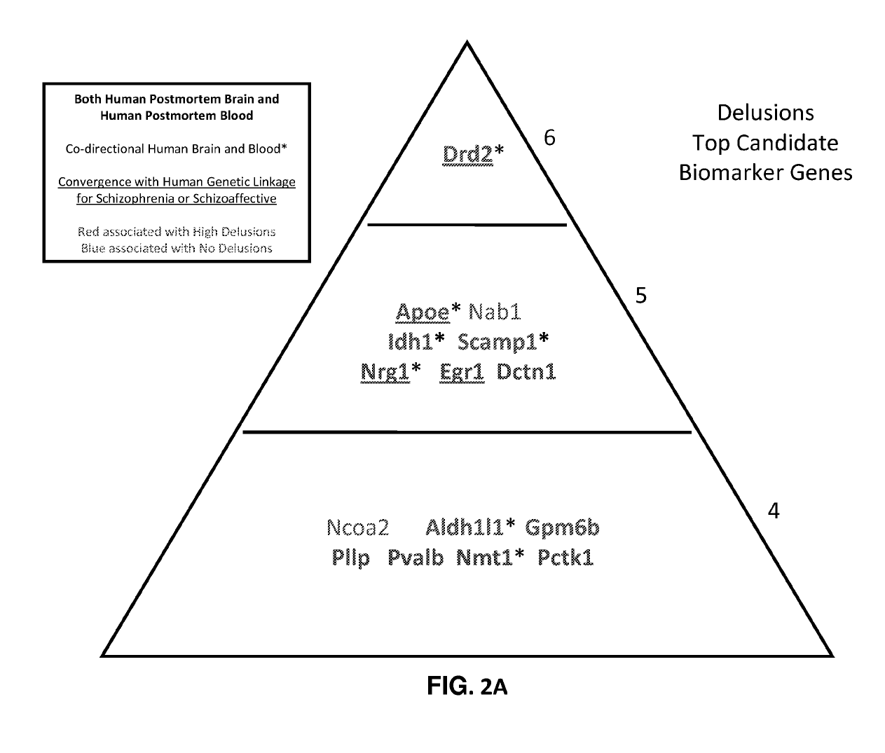

Human whole blood gene expression studies were initially carried out in a primary cohort of psychosis subjects. Whole blood was used as a way of minimizing potential artifacts related to sample handling and separation of individual cell types, and also as a way of having a streamlined approach that lends itself well to scalability and future large scale studies in the field. Genes that were differentially expressed in no symptoms vs. high symptoms subjects were compared with: 1) the results of the animal model brain and blood data, as well as 2) published human genetic linkage/association data, and 3) human postmortem brain data, as a way of cross-validating the findings, prioritizing them, and coming up with a short list of high probability candidate biomarker genes (FIGS. 1A and 2).

A focused approach was used for looking separately at two discrete quantitative phenotypic items (phenes), the Hallucinations item and the Delusions item from the PANSS. This approach avoids the issue of corrections for multiple comparisons that would arise if we were to look in a discovery fashion at multiple phenes in a comprehensive phenotypic battery (PhenoChipping) changed in relationship with all genes on a GeneChip microarray.

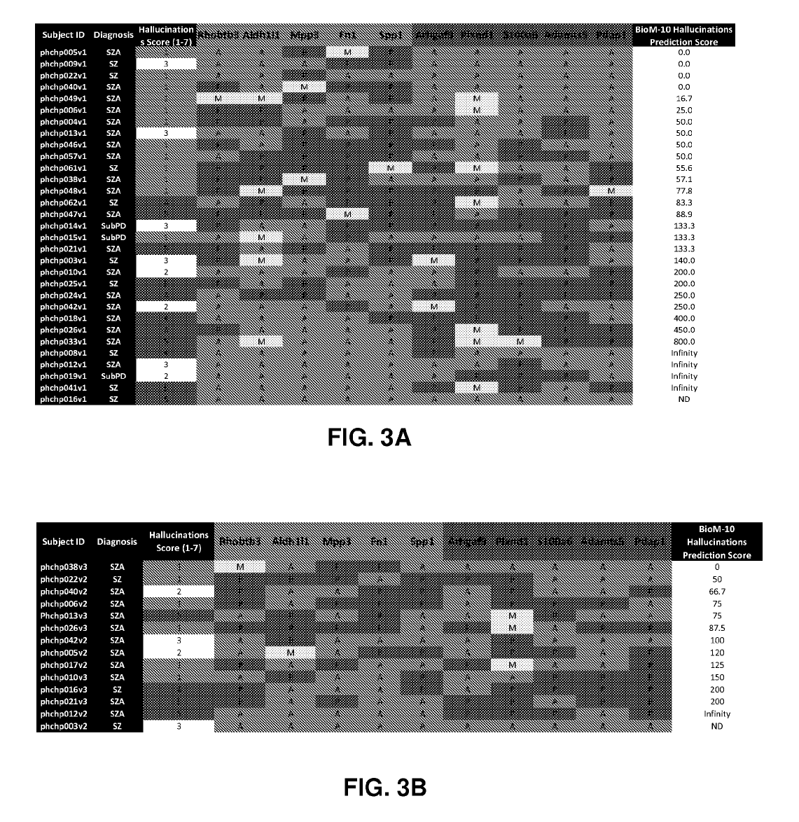

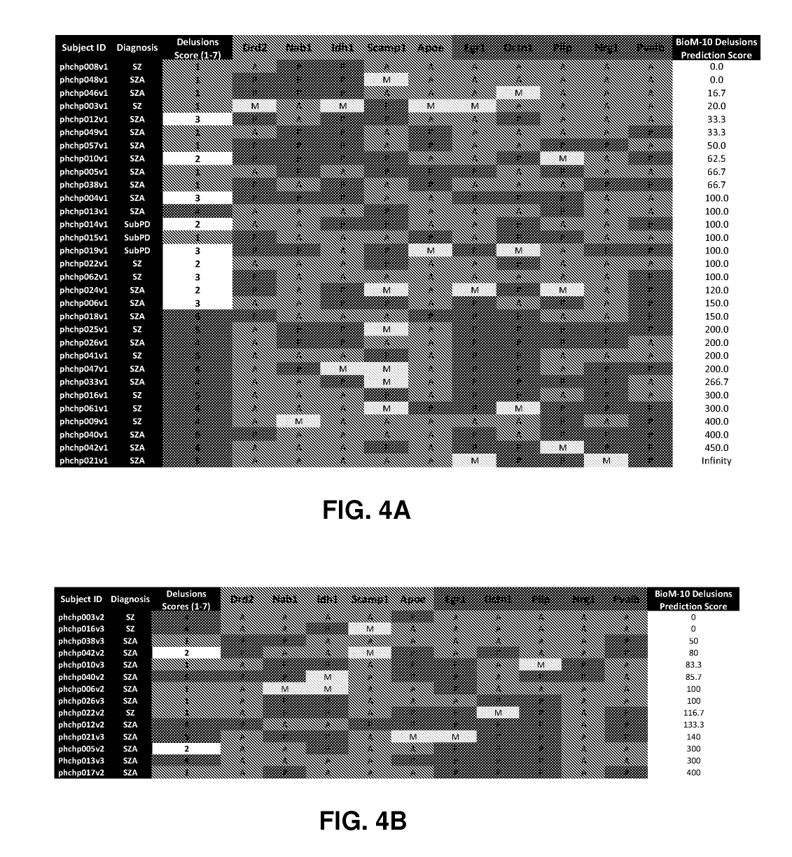

A panel of top candidate biomarker genes for hallucinations, respectively delusions state identified by the approach herein was then used to generate a prediction score for state (no symptoms vs. high symptoms). This prediction score was compared to the actual PANSS testing scores from psychosis subjects in the primary cohort (FIGS. 3A and 4A). The panels of biomarkers were examined and prediction scores in a separate cohort of psychotic disorders patients (FIG. 4C).

Sample size for human subjects (n=31 for the primary cohort, n=14 for the secondary cohort) is relatively small, but comparable to the size of cohorts for human postmortem brain gene expression studies. Live donor blood samples were studied instead of postmortem donor brains, with the advantage of better phenotypic characterization, more quantitative state information, and less technical variability. This approach also permits repeated intra-subject measures when the subject is in different psychosis states.

The experimental approach used in an embodiment for detecting gene expression changes relies on a standard methodology, Affymetrix GeneChip oligonucleotide microarrays. The analyses have been designed to minimize the likelihood of having false positives, even at the expense of potentially having false negatives, due to the high cost in time and resources of pursuing false leads. For the animal model work, using isogenic mouse strain affords us an ideal control baseline of saline injected animals for the drug-injected animals. Three independent de novo biological experiments were performed, at different times, with different batches of mice. This overall design is geared to factor out both biological and technical variability. It is to be noted that the concordance between reproducible microarray experiments using the latest generations of oligonucleotide microarrays and other methodologies such as quantitative PCR, with their own attendant technical limitations, is estimated to be over 90%. For the human blood samples differential gene expression analyses, which are the results of single biological experiments, it has to be noted that the approach used a very restrictive and technically robust, all or nothing induction of gene expression (change from Absent Call to Present Call). It is possible that not all biomarker genes for psychosis will show this complete induction related to state, but rather some will show modulation in gene expression levels, and are thus missed by an initial filtering. A classic differential expression analysis to identify additional possible candidate biomarkers (see Tables 6A and 6 B).

Moreover, given the genetic heterogeneity and variable environmental exposure, it is possible, that not all subjects will show changes in all the biomarker genes. Hence have two stringency thresholds were used: changes in 75% of subjects, and in 60% of subjects with no symptoms vs. high symptoms. Moreover, an approach described herein is predicated on the existence of multiple cross-validators for each gene that is called a candidate biomarker (FIG. 1A): 1) is it changed in human blood, 2) is it changed in animal model brain, 3) is it changed in animal model blood, 4) is it changed in postmortem human brain, and 5) does in map to a human genetic linkage locus. All these lines of evidence are the result of independent experiments. The virtues of this networked Bayesian approach are that, if one or another of the nodes (lines of evidence) becomes questionable/non-functional upon further evidence in the field, the network is resilient and maintains functionality. As more evidence emerges in the field for some of these genes, they will move up in the prioritization scoring. Using such an approach, a small number of genes were identified as likely candidate biomarkers, out of the over 40,000 transcripts (about half of which are detected as Present in each subject) measured by the microarrays that were used.

A validation of is the fact that the primary cohort-derived biomarker panels showed explanatory sensitivity and specificity, of a comparable nature, in the primary cohort. They also showed some predictive sensitivity and specificity in the second (replication) cohort, more so for hallucinations than for delusions. Thus, the approach of using two individual phenes reflecting internal subjective experiences (hallucinations, delusions) which are the hallmark of psychosis (as opposed to more complex and disease specific state/trait clinical instruments), and looking at extremes of state combined with robust differential expression based on A/P calls, and Convergent Functional Genomics prioritization, seems to be able to identify state biomarkers for psychosis. Nevertheless, a comparison with existing clinical rating scales, EEG and functional neuroimaging, as well as analysis of biomarker data using such instruments are also suitable for a way of delineating state vs. trait issues, diagnostic boundaries or lack thereof, and informing the design of clinical trials that may incorporate clinical and biomarker measures of response to treatment.

Human blood gene expression changes may be influenced by the presence or absence of both medications and drugs of abuse. That medications and drugs of abuse may have effects on mood state and gene expression is in fact being partially modeled in the mouse pharmacogenomic model, with clozapine and PCP treatments respectively. It is the association of blood biomarkers with psychosis state a primary goal of this study, regardless of the proximal causes, which could be diverse (see FIG. 1B), Candidate biomarkers at a protein level, in larger cohorts of both genders, in different age groups, and in theragnostic settings—measuring responses to specific treatments/medications are also analyzed.

Top candidate biomarker genes for hallucinations include four genes decreased in expression in high hallucinations states (Rhobtb3, Aldh111, Mpp3, Fn1), and two genes increased in high hallucinations states (Arhgef9, S100a6). Five of these genes have evidence of differential expression in human postmortem brains from schizophrenia patients.

Top candidate biomarker genes for delusions include eight genes decreased in expression in high delusions states (Drd2, ApoE, Nab1, Idh1, Scamp1, Ncoa2, Aldh111, Gpm6b), and eight genes increased in high delusions states (Nrg1, Egr1, Dctn1, Nmt1, Pllp, Pvalb, Nmt1, Pctk1). Fourteen of these genes have evidence of differential expression in human postmortem brains from schizophrenia patients.

It is intriguing that genes which have a well-established role in brain functioning should show changes in blood in relationship to psychiatric symptoms state (FIG. 2, Tables 3A and 3B), and moreover that the direction of change should be concordant with that reported in human postmortem brain studies. It is possible that trait promoter sequence mutations or epigenetic modifications influence expression in both tissues (brain and blood), and that state dependent transcription factor changes that modulate the expression of these genes may be contributory as well.

There are to date no clinical laboratory blood tests for psychotic disorders. A translational convergent approach to help identify and prioritize blood biomarkers for psychosis state is disclosed. Data demonstrate that blood biomarkers have the potential to offer an unexpectedly informative window into brain functioning and disease state. Panels of such biomarkers serve as a basis for objective clinical laboratory tests, a longstanding Holy Grail for psychiatry. Biomarker-based tests help with early intervention and prevention efforts, as well as monitoring response to various treatments. In conjunction with other clinical information, such tests play an important part in personalizing treatment to increase effectiveness and avoid adverse reactions—personalized medicine in psychiatry. Moreover, they have scientific use in combination with imaging studies (imaging genomics), and is useful to pharmaceutical companies engaged in new neuropsychiatric drug development efforts, at both a pre-clinical and clinical (Phase I, II and III) stages of the process.

In an embodiment, the 5 top scoring candidate biomarkers for high delusions and the 5 top scoring candidate biomarkers for low delusions, and doing the same for hallucinations, a panel of 10 biomarkers for delusions, and a panel of 10 biomarkers for hallucinations have been designed for diagnostic and predictive purposes. However, a panel may have more or less number of genes specified in this embodiment.

To test the predictive value of the panels (to be called the BioM-10 Delusions panel and BioM-10 Hallucinations panel), a cohort of 30 psychotic disorder datasets was analyzed, containing the datasets from which the candidate biomarker data was derived, as well as additional datasets of subjects with psychosis scores in the intermediate range (self-reported psychosis scores of 2 and 3) (Table 2). A prediction score for each subject was derived, based on the presence or absence of the 10 biomarker of the panel in their blood GeneChip data. Each of the 10 biomarkers gets a score of 1 if it is detected as Present (P) in the blood form that subject, 0.5 if it is detected as Marginally Present (M), and 0 if it is called Absent (A). The ratio of the sum of the high psychosis biomarker scores divided by the sum of the low psychosis biomarker scores is multiplied by 100, and provides a prediction score. If the ratio of high biomarker genes to low psychosis biomarker genes is 1, i.e. the two sets of genes are equally represented, the psychosis prediction score is 1×100=100. The higher this score, the higher the predicted likelihood that the subject will have a high psychosis symptoms score. The predictive score was compared with actual psychosis scores in the cohort of samples with a diagnosis of psychosis (n=30).

For example, suitable candidate biomarker genes include for example,

A prediction score above 100 had a 100% sensitivity and an 47.1% specificity for predicting a high delusions state. A prediction score below 100 had a 62.5% sensitivity and 84.2% specificity for predicting low delusions state (FIG. 3).

A prediction score above 100 had a 100% sensitivity and an 64.7% specificity for predicting a high hallucinations state. A prediction score below 100 had a 85.7% sensitivity and 88.9% specificity for predicting a low hallucinations state (FIG. 4).

The MIT/Broad Institute Connectivity Map13 was interrogated with a signature query composed of the genes in BioM-10 Delusions and BioM-10 Hallucinations panels of top biomarkers for low and high psychosis (FIG. 5). It was determined which drugs in the Connectivity Map database have similar effects on gene expression as the effects of high psychosis (delusions, respectively hallucinations) on gene expression, and which drugs have the opposite effect to high psychosis. As such, as part of the signature query, separately for delusions and hallucinations, the 5 biomarkers for high psychosis were considered as genes “Increased” by high psychosis, the 5 biomarkers for low psychosis were genes “Decreased” by high psychosis.

The interrogation revealed that deferoxamine had the most similar effects to high delusions, and sulindac the most similar effects to low delusions. For hallucinations, fluphenazine had the most similar effects to high hallucinations, and wortmaninn had the most similar effects to low hallucinations.

In an embodiment, a comprehensive analysis of: (i) fresh human blood gene expression data tied to illness state (quantitative measures of symptoms), (ii) cross-validation of blood gene expression profiling in conjunction with brain gene expression studies in animal models presenting key features of psychotic disorders, and (iii) integration of the results in the context of the available human genetic linkage/association and postmortem brain findings in the field is provided.

A panel of 289 biomarker genes for Delusions, and 138 biomarker genes for hallucinations were identified, as illustrated in an example described herein, is a suitable subset that is useful in diagnosing psychotic disorders. Larger subsets that includes for example, 300, 350, 400, 450, or 500 markers are also suitable. Smaller subsets that include high-value markers including about 2, 5, 10, 15, 20, 25, 50, 75, and 100 are also suitable. A variable quantitative scoring scheme can be designed using any standard algorithm, such as a variable selection or a subset feature selection algorithms can be used. Both statistical and machine learning algorithms are suitable in devising a frame work to identify, rank, and analyze association between marker data and phenotypic data (e.g., psychotic disorders).

A panel of 36 biomarkers, as illustrated in an example described herein, is a suitable subset that is useful in diagnosing a mood disorder. Larger subsets that includes for example, 150, 200, 250, 300, 350, 400, 450, 500, 600 or about 700 markers are also suitable. Smaller subsets that include high-value markers including about 2, 5, 10, 15, 20, 25, 50, 75, and 100 are also suitable. A variable quantitative scoring scheme can be designed using any standard algorithm, such as a variable selection or a subset feature selection algorithms can be used. Both statistical and machine learning algorithms are suitable in devising a frame work to identify, rank, and analyze association between marker data and phenotypic data (e.g., mood disorders).

In an embodiment, a prediction score for each subject is derived based on the presence or absence of e.g., 10 biomarkers of the panel in their blood. Each of the 10 biomarkers gets a score of 1 if it is detected as “present” (P) in the blood form that subject, 0.5 if it is detected as “marginally present” (M), and 0 if it is called “absent” (A). The ratio of the sum of the high mood biomarker scores divided by the sum of the low mood biomarker scores is multiplied by 100, and provides a prediction score. If the ratio of high biomarker genes to low mood biomarker genes is 1, i.e. the two sets of genes are equally represented, the mood prediction score is 1×100=100. The higher this score, the higher the predicted likelihood that the subject will have high mood. The predictive score was compared with actual self-reported mood scores in a primary cohort of subjects with a diagnosis of bipolar mood disorder. A prediction score of 100 and above had a 84.6% sensitivity and a 68.8% specificity for predicting high mood. A prediction score below 100 had a 76.9% sensitivity and 81.3% specificity for predicting low mood. The term “present” indicates that a particular biomarker is expressed to a detectable level, as determined by the technique used. For example, in an experiment involving a microarray or gene chip obtained from a commercial vendor Affymetrix (Santa Clara, Calif.), the embedded software rendered a “present” call for that biomarker. The term “present” refers to a detectable presence of the transcript or its translated protein/peptide and not necessarily reflects a relative comparison to for example, a sample from a normal subject. In other words, the mere presence or absence of a biomarker is assigned a value, e.g., 1 and a prediction score is calculated as described herein. The term “marginally present: refers to border line expression level that may be less intense than the “present” but statistically different from being marked as “absent” (above background noise), as determined by the methodology used.

In an embodiment, a prediction score based on differential expression (instead of “present”, “absent”) is used. For example, if a subject has a plurality of markers for high or low mood are differentially expressed, a prediction based on the differential expression of markers is determined. Differential expression of about 1.2 fold or 1.3 or 1.5 or 2 or 3 or 4 or 5-fold or higher for either increased or decreased levels can be used. Any standard statistical tool such as ANOVA is suitable for analysis of differential expression and association with high or low mood diagnosis or prediction.

A prediction based on the analysis of either high or low mood markers alone (instead of a ratio of high versus low mood markers) may also be practiced. If a plurality of high mood markers (e.g., about 6 out of 10 tested) are differentially expressed to a higher level compared to the low mood markers (e.g., 4 out of 10 tested), then a prediction or diagnosis of high mood status can be made by analyzing the expression levels of the high mood markers alone without factoring the expression levels of the low mood markers as a ratio.

In an embodiment, a detection algorithm uses probe pair intensities to generate a detection p-value and assign a Present, Marginal, or Absent call. Each probe pair in a probe set is considered as having a potential vote in determining whether the measured transcript is detected (Present) or not detected (Absent). The vote is described by a value called the Discrimination score [R]. The score is calculated for each probe pair and is compared to a predefined threshold Tau. Probe pairs with scores higher than Tau vote for the presence of the transcript. Probe pairs with scores lower than Tau vote for the absence of the transcript. The voting result is summarized as a p-value. The greater the number of discrimination scores calculated for a given probe set that are above Tau, the smaller the p-value and the more likely the given transcript is truly Present in the sample. The p-value associated with this test reflects the confidence of the Detection call.

Regarding detection p-value, a two-step procedure determines the Detection p-value for a given probe set. The Discrimination score [R] is calculated for each probe pair and the discrimination scores are tested against the user-definable threshold Tau. The detection Algorithm assesses probe pair saturation, calculates a Detection p-value, and assigns a Present, Marginal, or Absent call. In an embodiment, the default thresholds of the Affymetrix MAS 5 software were used.

In spiking experiments by the manufacturer to establish default thresholds (adding of known quantities of test transcripts to a mixture, to measure the sensitivity of the Affymetrix MAS 5 detection algorithm) 80% of spiked transcripts are called Present at a concentration of 1.5 pM. This concentration corresponds to approximately one transcript in 100,000 or 3.5 copies per cell. The false positive rate of making a Present call was roughly 10%, as noted by 90% of the transcripts being called Absent when not spiked into the sample (0 pM concentration).

The term “predictive” or the term “prognostic” does not imply 100% predictive ability. The use of these terms indicates that subjects with certain characteristics are more likely to experience a clinically positive outcome than subjects who do not have such characteristics. For example, characteristics that determine the outcome include one or more of the biomarkers for psychosis disclosed herein. Certain conditions are identified herein as associated with an increased likelihood of a clinically positive outcome, e.g., biomarkers for delusions and the absence of such conditions or markers will be associated with a reduced likelihood of a clinically positive outcome.

The phrase “clinically positive outcome” refers to biological or biochemical or physical or physiological responses to treatments or therapeutic agents that are generally prescribed for that condition compared to a condition would occur in the absence of any treatment. A “clinically positive outcome” does not necessarily indicate a cure, but could indicate a lessening of symptoms experienced by a subject.

The terms “marker” and “biomarker” are synonymous and as used herein, refer to the presence or absence or the levels of nucleic acid sequences or proteins or polypeptides or fragments thereof to be used for associating or correlating a phenotypic state. A biomarker includes any indicia of the level of expression of an indicated marker gene. The indicia can be direct or indirect and measure over- or under-expression of the gene given the physiologic parameters and in comparison to an internal control, normal tissue or another phenotype. Nucleic acids or proteins or polypeptides or portions thereof used as markers are contemplated to include any fragments thereof, in particular, fragments that can specifically hybridize with their intended targets under stringent conditions and immunologically detectable fragments. One or more markers may be related. Marker may also refer to a gene or DNA sequence having a known location on a chromosome and associated with a particular gene or trait. Genetic markers associated with certain diseases or for pre-disposing disease states can be detected in the blood and used to determine whether an individual is at risk for developing a disease. Levels of gene expression and protein levels are quantifiable and the variation in quantification or the mere presence or absence of the expression may also serve as markers. Using proteins/peptides as biomarkers can include any method known in the art including, without limitation, measuring amount, activity, modifications such as glycosylation, phosphorylation, ADP-ribosylation, ubiquitination, etc., immunohistochemistry (IHC).

As used herein, “array” or “microarray” refers to an array of distinct polynucleotides, oligonucleotides, polypeptides, or oligopeptides synthesized on a substrate, such as paper, nylon, or other type of membrane, filter, chip, glass slide, or any other suitable solid support. Arrays also include a plurality of antibodies immobilized on a support for detecting specific protein products. There are several microarrays that are commercially available. A microarray may include one or more biomarkers disclosed herein. A panel of about 20 biomarkers as nucleic acid fragments can be included in an array. The nucleic acid fragments may include oligonucleotides or amplified partial or complete nucleotide sequences of the biomarkers. The term “consisting essentially of” generally refers to a collection of markers that substantially affects the determination of the disorder and may include other components such as controls. For example, top biomarkers from Tables 3A-B may be considered as a subset of markers that determine the most association.

In an embodiment, the microarray is prepared and used according to the methods described in U.S. Pat. No. 5,837,832, Chee et al.; PCT application WO95/11995, Chee et al.; Lockhart et al., 1996. Nat Biotech., 14:1675-80; and Schena et al., 1996. Proc. Natl. Acad. Sci. 93:10614-619, all of which are herein incorporated by reference to the extent they relate to methods of making a microarray. Arrays can also be produced by the methods described in Brown et al., U.S. Pat. No. 5,807,522. Arrays and microarrays may be referred to as “DNA chips” or “protein chips.”

A variety of clustering methods are available for microarray-based gene expression analysis. See for example, Shamir & Sharan (2002) Algorithmic approaches to clustering gene expression data. In Current Topics In Computational Molecular Biology (Edited by: Jiang T, Xu Y, Smith T). 2002, 269-300; Tamames et al., (2002): Bioinformatics methods for the analysis of expression arrays: data clustering and information extraction, J Biotechnol, 98:269-283.

“Therapeutic agent” means any agent or compound useful in the treatment, prevention or inhibition of psychosis or a psychosis-related disorder.

The term “condition” refers to any disease, disorder or any biological or physiological effect that produces unwanted biological effects in a subject.

The term “subject” refers to an animal, or to one or more cells derived from an animal. The animal may be a mammal including humans. Cells may be in any form, including but not limited to cells retained in tissue, cell clusters, immortalized cells, transfected or transformed cells, and cells derived from an animal that have been physically or phenotypically altered.

Any body fluid of an animal can be used in the methods of the invention. Suitable body fluids include a blood sample (e.g., whole blood, serum or plasma), urine, saliva, cerebrospinal fluid, tears, semen, and vaginal secretions. Also, lavages, tissue homogenates and cell lysates can be utilized.

Many different methods can be used to determine the levels of markers. For example, protein arrays, protein chips, cDNA microarrays or RNA microarrays are suitable. More specifically, one of ordinary skill in the art will appreciate that in one example, a microarray may comprise the nucleic acid sequences representing genes listed in Table 1. For example, functionality, expression and activity levels may be determined by immunohistochemistry, a staining method based on immunoenzymatic reactions uses monoclonal or polyclonal antibodies to detect cells or specific proteins. Typically, immunohistochemistry protocols include detection systems that make the presence of markers visible (to either the human eye or an automated scanning system), for qualitative or quantitative analyses. Mass-spectrometry, chromatography, real-time PCR, quantitative PCR, probe hybridization, or any other analytical method to determine expression levels or protein levels of the markers are suitable. Such analysis can be quantitative and may also be performed in a high-through put fashion. Cellular imaging systems are commercially available that combine conventional light microscopes with digital image processing systems to perform quantitative analysis on cells and tissues, including immunostained samples. (See e.g. the CAS-200 System (Becton, Dickinson & Co.)). Some other examples of methods that can be used to determine the levels of markers include immunohistochemistry, automated systems, quantitative IHC, semi-quantitative IHC and manual methods. Other analytical systems include western blotting, immunoprecipitation, fluorescence in situ hybridization (FISH), and enzyme immunoassays.

The term “diagnosis”, as used in this specification refers to evaluating the type of disease or condition from a set of marker values and/or patient symptoms where the subject is suspected of having a disorder. This is in contrast to disease predisposition, which relates to predicting the occurrence of disease before it occurs, and the term “prognosis”, which is predicting disease progression in the future based on the marker levels/patterns.

The term “correlating,” as used in this specification refers to a process by which one or more biomarkers are associated to a particular disease state, e.g., mood disorder. In general, identifying such correlation or association involves conducting analyses that establish a statistically significant association- and/or a statistically significant correlation between the presence (or a particular level) of a marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed.

This relationship or association can be determined by comparing biomarker levels in a subject to levels obtained from a control population, e.g., positive control—diseased (with symptoms) population and negative control—disease-free (symptom-free) population. The biomarkers disclosed herein provide a statistically significant correlation to diagnosis at varying levels of probability. Subsets of markers, for example a panel of about 20 markers, each at a certain level range which are a simple threshold, are said to be correlative or associative with one of the disease states. Such a panel of correlated markers can be then be used for disease detection, diagnosis, prognosis and/or treatment outcome. Preferred methods of correlating markers is by performing marker selection by any appropriate scoring method or by using a standard feature selection algorithm and classification by known mapping functions. A suitable probability level is a 5% chance, a 10% chance, a 20% chance, a 25% chance, a 30% chance, a 40% chance, a 50% chance, a 60% chance, a 70% chance, a 75% chance, a 80% chance, a 90% chance, a 95% chance, and a 100% chance. Each of these values of probability is plus or minus 2% or less. A suitable threshold level for markers of the present invention is about 25 pg/mL, about 50 pg/mL, about 75 pg/mL, about 100 pg/mL, about 150 pg/mL, about 200 pg/mL, about 400 pg/mL, about 500 pg/mL, about 750 pg/mL, about 1000 pg/mL, and about 2500 pg/mL.

Prognosis methods disclosed herein that improve the outcome of a disease by reducing the increased disposition for an adverse outcome associated with the diagnosis. Such methods may also be used to screen pharmacological compounds for agents capable of improving the patient's prognosis, e.g., test agents for mood disorders.

The analysis of a plurality of markers, for example, a panel of about 20 or 10 markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples may allow the identification of changes in marker levels over time, within a period of interest, or in response to a certain treatment.

In another embodiment, a kit for the analysis of markers includes for example, devises and reagents for the analysis of at least one test sample and instructions for performing the assay. Optionally, the kits may contain one or more means for using information obtained from marker assays performed for a marker panel to diagnose psychosis. Probes for markers, marker antibodies or antigens may be incorporated into diagnostic assay kits depending upon which markers are being measured. A plurality of probes may be placed in to separate containers, or alternatively, a chip may contain immobilized probes. In an embodiment, another container may include a composition that includes an antigen or antibody preparation. Both antibody and antigen preparations may preferably be provided in a suitable titrated form, with antigen concentrations and/or antibody titers given for easy reference in quantitative applications.

The kits may also include a detection reagent or label for the detection of specific reaction between the probes provided in the array or the antibody in the preparation for immunodetection. Suitable detection reagents are well known in the art as exemplified by fluorescent, radioactive, enzymatic or otherwise chromogenic ligands, which are typically employed in association with the nucleic acid, antigen and/or antibody, or in association with a secondary antibody having specificity for first antibody. Thus, the reaction is detected or quantified by means of detecting or quantifying the label. Immunodetection reagents and processes suitable for application in connection with the novel methods of the present invention are generally well known in the art.

The reagents may also include ancillary agents such as buffering agents and protein stabilizing agents, e.g., polysaccharides and the like. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, and the like.

In some embodiments, the methods of correlating biomarkers with treatment regimens can be carried out using a computer database. Computer-assisted methods of identifying a proposed treatment for mood disorders are suitable. The method involves the steps of (a) storing a database of biological data for a plurality of patients, the biological data that is being stored including for each of said plurality of patients (i) a treatment type, (ii) at least one marker associated with a mood disorder and (iii) at least one disease progression measure for the mood disorder from which treatment efficacy can be determined; and then (b) querying the database to determine the dependence on the marker of the effectiveness of a treatment type in treating the mood disorder, to thereby identify a proposed treatment as an effective treatment for a subject carrying the marker correlated with the mood disorder.

In an embodiment, treatment information for a patient is entered into the database (through any suitable means such as a window or text interface), marker information for that patient is entered into the database, and disease progression information is entered into the database. These steps are then repeated until the desired number of patients has been entered into the database. The database can then be queried to determine whether a particular treatment is effective for patients carrying a particular marker, not effective for patients carrying a particular marker, and the like. Such querying can be carried out prospectively or retrospectively on the database by any suitable means, but is generally done by statistical analysis in accordance with known techniques, as described herein.

### EXAMPLES

The following examples are to be considered as exemplary and not restrictive or limiting in character and that all changes and modifications that come within the spirit of the disclosure are desired to be protected.

### Example 1

### Experimental Framework for Identification of Biomarkers Used in Diagnosis of Psychotic Disorders

Gene expression changes in specific brain regions and blood from a pharmacogenomic animal model developed in the group were used as cross-validators to help with the identification of potential human blood biomarkers. Pharmacogenomic mouse model of relevance to bipolar disorder consists of treatments with an agonist of the illness/psychosis-mimicking drug (PCP) and an antagonist of the illness/bipolar disorder-treating drug (clozapine) 4. The pharmacogenomic approach is a tool for tagging genes that may have pathophysiological relevance.

Human blood gene expression studies were carried out in a cohort of psychotic disorders subjects. Genes that were differentially expressed in low psychosis vs. high psychosis subjects were compared with: 1) the results of animal model brain and blood data, as well as 2) human genetic linkage/association data, and 3) human postmortem brain data, as a way of cross-validating the findings, prioritizing them, and identifying a short list of high probability candidate biomarker genes (FIG. 1A and FIG. 3).

PANSS-P1 score for Delusions, and P3 score for Hallucinations were used. This approach avoids the issue of corrections for multiple comparisons that would arise if discovery at multiple phenes was considered in a comprehensive phenotypic battery changed in relationship with all genes on a GeneChip microarray. Larger sample cohorts would be needed for the latter approach.

In an aspect, the sample size for human subjects (n=30 for the psychotic disorders cohort) is relatively small, but comparable to the size of cohorts for human postmortem brain gene expression studies. Live donor blood samples were studied instead of postmortem donor brains, with the advantage of better phenotypic characterization, more quantitative state information, and less technical variability.

Some of the datasets were derived from subjects that were sampled repeatedly, at three months intervals (Table 2). A total of 21 unique subjects were used and 2 of them were sampled three times, and 5 of them were sampled twice. However, this reduction in genetic background diversity may be advantageous in terms of analysis of state related markers, which is an important objective. In mouse models, the isogenic strain background is viewed as advantageous in terms of reducing noise and providing power to pharmacogenomic analyses.

In an aspect, the experimental approach for detecting gene expression changes relies on a chip methodology, Affymetrix GeneChip oligonucleotide microarrays. It is possible that some of the gene expression changes detected from a single biological experiment, with a one-time assay with this technology, are biological or technical artifacts. The analyses are designed to minimize the likelihood of having false positives, even at the expense of potentially having false negatives, due to the high cost in time and resources of pursuing false leads. For the animal model work, using isogenic mouse strain affords us an ideal control baseline of saline injected animals for the drug-injected animals. Three independent de novo biological experiments were performed at different times, with different batches of mice. This overall design is geared to factor out both biological and technical variability. It is to be noted that the concordance between reproducible microarray experiments using the latest generations of oligonucleotide microarrays and other methodologies such as quantitative PCR, with their own attendant technical limitations, is estimated to be over 90%. For the human blood samples gene expression analyses, a very restrictive approach was used—all or nothing induction of gene expression (change from Absent Call to Present Call). Moreover, given the genetic heterogeneity and variable environmental exposure, it is possible, indeed likely, that not all subjects will show changes in all the biomarker genes. Therefore, two stringency thresholds were used: changes in 75% of subjects, and in 60% of subjects with low psychosis vs. high psychosis. Moreover, the approach, as described above, is predicated on the existence of multiple cross-validators for each gene that is called a candidate biomarker (FIG. 1B): 1) is it changed in human blood? 2) is it changed in animal model brain? 3) is it changed in animal model blood? 4) is it changed in postmortem human brain? and 5) does in map to a human genetic linkage locus? All these lines of evidence are the result of independent experiments.

Human blood gene expression changes may be influenced by the presence or absence of both medications and drugs of abuse. That medications and drugs of abuse may have effects on psychosis state and gene expression is being partially modeled in the mouse pharmacogenomic model, with clozapine and PCP treatments respectively.

A panel of top candidate biomarker genes for psychosis state identified by the methods disclosed herein was then used to generate a prediction score for psychosis state (low psychosis symptoms vs. high psychosis symptoms). This prediction score was compared to the actual psychosis scores from psychotic disorder subjects (FIGS. 4A and B). Methods disclosed herein narrow the over 40,000 genes and ESTs (transcript variants) present on the Affymetix Human Genome U133 Plus 2.0 GeneChip, about half of which are detected as Present in each blood sample, to a panel of 10 high probability biomarker genes, which shows surprisingly robust predictive power.

In an aspect, a panel of biomarkers for delusions include a gene associated with low delusions scores (MOBP) and three genes associated with high delusions scores (NRG1, GPM6B, and TPM2). In an aspect, a panel of biomarkers for hallucinations, three genes are associated with high hallucinations scores (TNIK, HSD17B12 and TPM2). These genes were selected as having a line of evidence CFG score of higher than 5 (Table 4 and 5, and FIGS. 2A and 2B). That means, in addition to the human blood data, these genes have at least two other independent lines of evidence implicating them in psychotic disorders. All these genes have evidence of differential expression in human postmortem brains from schizophrenia patients. NRG1 (neuroregulin 1) has been implicated in the pathogenesis of schizophrenia by multiple genetic and neurobiological studies.

It is intriguing that genes which have a well-established role in brain functioning should show changes in blood in relationship to psychiatric symptoms state (FIG. 2, Table 4-5 and Table 6), and moreover that the direction of change should be concordant with that reported in human postmortem brain studies. It is possible that trait promoter sequence mutations or epigenetic modifications influence expression in both tissues (brain and blood), and that state dependent transcription factor changes that modulate the expression of these genes may be contributory as well.

Data provided herein suggest that genes involved in brain infrastructure changes (myelin, growth factors) are prominent players in psychotic disorders, and are reflected in the blood profile. Myelin abnormalities have emerged as a common if perhaps non-specific denominator across a variety of neuropsychiatric disorders. Data regarding cytoskeleton regulating genes (TPM2 and TNIK) changes may provide evidence for a novel and previously underappreciated mechanism for schizophrenia pathophysiology. HSD17B12 (17-beta-hydroxysteroid dehydrogenase) is an enzyme involved in estrogen formation. It is increased in the human blood data in high delusions states, as well as increased in human postmortem brain from schizophrenics and in the animal model brain data. Weather these changes are etiopathogenic, compensatory mechanisms, side-effects of medications or results of illness-induced lifestyle changes (FIG. 1B) is an intriguing area.

The fact that most of the top genes identified are associated with high psychosis states as opposed to low psychosis states (FIG. 2 and Table 4-5) may suggest that co-morbid stress—more prevalent in high psychosis than in low/no psychosis—is a factor in the richness of blood gene expression results, as part of a neuro-endocrine-immunological axis. The higher sensitivity than specificity of the test for high psychosis state may reflect this preponderance of candidate biomarker genes for high psychosis state identified relative to candidate biomarker for low psychosis state. The test shows lower sensitivity but higher specificity for low psychosis state.

Of note, some of the other top candidate genes identified have no previous evidence for involvement in psychosis other than them being mapped to schizophrenia genetic linkage loci (Table 4-5), and thus constitute novel candidate genes for schizophrenia. They are useful for whole—genome association studies of schizophrenia. It is possible that the composition of top biomarker panels for psychosis will be refined or changed for different sub-populations. That being said, it is likely that a large number of the biomarkers that would be of use in different panels and permutations are already present in the complete list of top candidate biomarkers (n=289 top candidate biomarker genes for Delusions, and n=138 top candidate biomarker genes for Hallucinations). (Tables 10-11).

The interrogation of the MIT/Broad Institute Connectivity Map 13 with a signature query composed of the genes in the BioM-10 Delusions and BioM-10 Hallucinations panels of top biomarkers revealed that deferoxamine had the most similar effects to high delusions, and sulindac the most similar effects to low delusions. For hallucinations, fluphenazine had the most similar effects to high hallucinations, and wortmaninn had the most similar effects to low hallucinations (FIGS. 4A and 4B).

Deferoxamine is a medication used clinically to treat iron overload states. Oligodendrocyte progenitors are highly susceptible to oxidative stress due to their limited content of antioxidants and high iron levels. Iron plays a central role in the toxicity of dopamine to oligodendrocyte progenitors. Dopamine induces accumulation of superoxide, membrane damage and loss in cell viability. The iron chelator deferoxamine reduces superoxide accumulation. Desferrioxamine administration in mice caused a reduction in severity of physical dependence to alcohol. Deferoxamine also increases the production of neurons from neural stem/progenitor cells, and showed neuroprotective properties in ischemia states. These observations indicate that deferoxamine activates cellular mechanisms and programs of gene expression that have cell survival and protective effects. Sulindac, a non-steroidal inflammatory drug, has been shown to inhibit liver tryptophan 2,3-dioxygenase activity, a rate-limiting enzyme in tryptophan catabolism, and consequently alter brain neurotransmitter levels, resulting in an increase in serotonin levels and decrease in dopamine levels in rats. Taken together, these observations indicate that high delusions are associated with a program of gene expression reflective of a neurotrophic, high dopamine state.

Fluphenazine is a typical (first-generation) anti-psychotic. Wortmannin is a phosphoinositide-3′ kinase (PI3K) inhibitor. The PI3K pathway is thought to be hypoactive in schizophrenia, suggesting that wortmannin has a schziophrenogenic effect. Results demonstrate that the gene expression patterns seen with hallucinations may be reflective of a medication effect in those severely psychotic patients.

This connectivity map analysis with the BioM-10 psychosis panels genes provides an interesting external biological cross-validation for the internal consistency of the biomarker approach, as well as illustrates the utility of the Connectivity Map for non-hypothesis driven identification of novel drug treatments and interventions.

More profoundly, these results, taken together with candidate biomarker genes results and biological roles categories (3A and 3B), are consistent with a developmental model for genes involved in psychosis.

There are to date no clinical laboratory blood tests for psychosis. A translational convergent approach to help identify blood biomarkers of psychosis symptoms (delusions, hallucinations) state is proposed herein. Blood biomarkers have the potential to offer an unexpectedly informative window into brain functioning and disease state. Panels of such biomarkers serve as a basis for objective clinical laboratory tests.

Any number of biomarkers can be used as a panel for diagnosis. The panel may contain equal number of biomarkers for delusions and hallucinations. The panel may be tested as a microarray or as any form of diagnostic analysis.

Thus, the biomarkers identified herein provide quantitative tools for predicting disease states/conditions in subjects suspected of having a psychotic disorder or in any individual for psychiatric evaluation.

Human subjects: Data from two cohorts of patients are presented herein. One cohort included 31 different subjects with psychotic disorders (schizophrenia, schizoaffective disorder and substance induced psychosis), from which the primary biomarker data was derived, from testing done at their first visit (v1). A second (replication) cohort consists of 14 subjects from the first cohort, tested 3 moths (v2) or 6 months (v3) later. The diagnosis is established by a structured clinical interview-Diagnostic Interview for Genetic Studies (DIGS), which has details on the course of illness and phenomenology, and is the scale used by the Genetics Initiative Consortia for both Bipolar Disorder and Schizophrenia.

Subjects included men and women over 18 years of age. Subjects were recruited from the patient population at the Indianapolis Va. Medical Center, the Indiana University School of Medicine, as well as various facilities that serve people with mental illnesses in Indiana. A demographic breakdown is shown in Table 1. Initial studies were focused primarily on an age-matched male population, due to the demographics of the catchment area (primarily male in a VA Medical Center), and to minimize any potential gender-related state effects on gene expression, which would have decreased the discriminative power of the analysis given a relatively small sample size. The subjects were recruited largely through referrals from care providers, the use of brochures left in plain sight in public places and mental health clinics, and through word of mouth. Subjects were excluded if they had significant medical or neurological illness or had evidence of active substance abuse or dependence. All subjects understood and signed informed consent forms detailing the research goals, procedure, caveats and safeguards. Subjects completed diagnostic assessments (DIGS), and then a psychosis rating scale (Positive and Negative Symptom Scale—PANSS) at the time of blood draw. 10 cc of whole blood were collected in two RNA-stabilizing PAXgene tubes, labeled with an annonymized ID number, and stored at −80 C in a locked freezer (Revco) until the time of future processing.

Human blood gene expression experiments and analysis: RNA extraction: 2.5-5 ml of whole blood was collected into each PaxGene tube by routine venipuncture. PaxGene tubes contain proprietary reagents for the stabilization of RNA. The cells from whole blood will be concentrated by centrifugation, the pellet washed, resuspended and incubated in buffers containing Proteinase K for protein digestion. A second centrifugation step is done to remove residual cell debris. After the addition of ethanol for an optimal binding condition the lysate is applied to a silica-gel membrane/column. The RNA bound to the membrane as the column is centrifuged and contaminants are removed in three wash steps. The RNA is then eluted using DEPC-treated water.

Globin reduction: To remove globin mRNA, total RNA from whole blood is mixed with a biotinylated Capture Oligo Mix that is specific for human globin mRNA. The mixture is then incubated for 15 min to allow the biotinylated oligonucleotides to hybridize with the globin mRNA. Streptavidin Magnetic Beads are then added, and the mixture is incubated for 30 min. During this incubation, streptavidin binds the biotinylated oligonucleotides, thereby capturing the globin mRNA on the magnetic beads. The Streptavidin Magnetic Beads are then pulled to the side of the tube with a magnet, and the RNA, depleted of the globin mRNA, is transferred to a fresh tube. The treated RNA is further purified using a rapid magnetic bead-based purification method. This consists of adding an RNA Binding Bead suspension to the samples, and using magnetic capture to wash and elute the GLOBINclear RNA.

Sample Labeling: Sample labeling is performed using the Ambion MessageAmp II-BiotinEnhanced aRNA amplification kit. The procedure is briefly outlined below and involves the following steps:

1. Reverse Transcription to Synthesize First Strand cDNA is primed with the T7 Oligo(dT) Primer to synthesize cDNA containing a T7 promoter sequence.

2. Second Strand cDNA Synthesis converts the single-stranded cDNA into a double-stranded DNA (dsDNA) template for transcription. The reaction employs DNA Polymerase and RNase H to simultaneously degrade the RNA and synthesize second strand cDNA.

3. cDNA Purification removes RNA, primers, enzymes, and salts that would inhibit in vitro transcription.

4. In Vitro Transcription to Synthesize aRNA with Biotin-NTP Mix generates multiple copies of biotin-modified aRNA from the double-stranded cDNA templates; this is the amplification step.

5. aRNA Purification removes unincorporated NTPs, salts, enzymes, and inorganic phosphate to improve the stability of the biotin-modified aRNA.

Microarrays: Biotin labeled aRNA are hybridized to Affymetrix HG-U133 Plus 2.0 GeneChips according to manufacturer's protocols http://www.affymetrix.com/support/technical/manual/expression_manual.affx. All GAPDH 3′/5′ ratios should be less than 2.0 and backgrounds under 50. Arrays are stained using standard Affymetrix protocols for antibody signal amplification and scanned on an Affymetrix GeneArray 2500 scanner with a target intensity set at 250. Present/Absent calls are determined using GCOS software with thresholds set at default values.

Analysis: The subject's psychosis scores at time of blood collection, specifically the scores for hallucinations (from 1—no symptoms to 7—extreme symptoms) and the scores for delusions (1 to 7), obtained from a PANSS scale were used (Table 1). Only at all or nothing gene expression differences were considered that are identified by Absent (A) vs. Present (P) Calls in the Affymetrix MAS software. Genes were classified whose expression is detected as Absent in the Low Psychosis subjects (score of 1 on a scale of 1 to 7) and detected as Present in the High Psychosis subjects (score of 4 or above, on a scale of 1 to 7), as being candidate biomarker genes for psychosis (specifically for delusions or hallucinations). Conversely, genes whose expression are detected as Present in the Low Psychosis subjects and Absent in the High Psychosis subjects are being classified as candidate biomarker genes for low psychosis.

Two thresholds were used for analysis of gene expression differences between low psychosis and high psychosis (Table 3). First, a high threshold was used, with at least 75% of subjects in the cohort showing a change in expression from Absent to Present between low and high psychosis (reflecting an at least 3 fold psychosis state related enrichment of the genes thus filtered). A low threshold was also used, with at least 60% of subjects in the cohort showing a change in expression from Absent to Present between low and high psychosis (reflecting an at least 1.5 fold psychosis state related enrichment of the genes thus filtered).

Animal model data: Schizophrenia pharmacogenomic model includes phencyclidine (PCP) and clozapine treatments in mice.

All experiments were performed with male C57/BL6 mice, 8 to 12 weeks of age, obtained from Jackson Laboratories (Bar Harbor, Me.), and acclimated for at least two weeks in the animal facility prior to any experimental manipulation. Mice were treated by intraperitoneal injection with either single-dose saline PCP (7.5 m\tilde{g}/kg), clozapine (2.5 m\tilde{g}/kg), or a combination of PCP and clozapine (7.5 m\tilde{g}/k\tilde{g} and 2.5 m\tilde{g}/kg). Three independent de novo biological experiments were performed at different times. Each experiment consisted of three mice per treatment condition, for a total of 9 mice per condition across the three experiments.

Mouse Blood collection: Twenty-four hours after drug administration, following the 24 hour time-point behavioral test, the mice were decapitated to harvest blood. The headless mouse body was put over a glass funnel coated with heparin and approximately 1 ml of blood/mouse was collected into a PAXgene blood RNA collection tubes, BD diagnostic (VWR.com). The Paxgene blood vials were stored in −4° C. overnight, and then at −80° C. until future processing for RNA extraction.

RNA extraction and microarray work: Standard techniques were used to obtain total RNA (22 gauge syringe homogenization in RLT buffer) and to purify the RNA (RNeasy mini kit, Qiagen, Valencia, Calif.) from micro-dissected mouse brain regions. For the human and whole mouse blood RNA extraction, PAXgene blood RNA extraction kit (PreAnalytiX, a QIAGEN/BD company) was used, followed by GLOBINclear™—Human or GLOBINclear™—Mouse/Rat (Ambion/Applied Biosystems Inc., Austin, Tex.) to remove the globin mRNA. All the methods and procedures were carried out as per manufacturer's instructions. The quality of the total RNA was confirmed using an Agilent 2100 Bioanalyzer (Agilent Technologies, Palo Alto, Calif.). The quantity and quality of total RNA was also independently assessed by 260 nm UV absorption and by 260/280 ratios, respectively (Nanodrop spectrophotometer). Starting material of total RNA labeling reactions was kept consistent within each independent microarray experiment.

For the all the mouse analysis, blood or brain tissues regions from 3 mice were pooled for each experimental condition, and equal amounts of total RNA extracted from tissue samples or blood was used for labeling and microarray assays. Mouse Genome 430 2.0 arrays (Affymetrix, Santa Clara, Calif.) were used. The GeneChip Mouse Genome 430 2.0 Array contains over 45,000 probe sets that analyze the expression level of over 39,000 transcripts and variants from over 34,000 well-characterized mouse genes. For the human work, we used Affymetrix Human Genome U133 Plus 2.0 GeneChip with over 40,000 genes and ESTs. Standard Affymetrix protocols were used to reverse transcribe the messenger RNA and generate biotinlylate cRNA. The amount of cRNA used to prepare the hybridization cocktail was kept constant intra-experiment. Samples were hybridized at 45° C. for 17 hours under constant rotation. Arrays were washed and stained using the Affymetrix Fluidics Station 400 and scanned using the Affymetrix Model 3000 Scanner controlled by GCOS software. All sample labeling, hybridization, staining and scanning procedures were carried out as per manufacturer's recommendations.

All arrays were scaled to a target intensity of 1000 using Affymetrix MASv 5.0 array analysis software. Quality control measures including 3′/5′ ratios for GAPDH and beta-actin, scaling factors, background, and Q values were within acceptable limits.

Microarray data analysis: Data analysis was performed using Affymetrix Microarray Suite 5.0 software (MAS v5.0). Default settings were used to define transcripts as present (P), marginal (M), or absent (A). A comparison analysis was performed for each drug treatment, using its corresponding saline treatment as the baseline. “Signal,” “Detection,” “Signal Log Ratio,” “Change,” and “Change p-value,” were obtained from this analysis. Only transcripts that were called Present in at least one of the two samples (saline or drug) intra-experiment, and that were reproducibly changed in the same direction in at least two out of three independent experiments, were analyzed further.

Cross-validation and integration: Convergent Functional Genomics: Gene identification The identities of transcripts were established using NetAFFX (Affymetrix, Santa Clara, Calif.), and confirmed by cross-checking the target mRNA sequences that had been used for probe design in the Mouse Genome 430 2.0 Array GeneChip® or the Affymetrix Human Genome U133 Plus 2.0 GeneChip® with the GenBank database. Where possible, identities of ESTs were established by BLAST searches of the nucleotide database. A National Center for Biotechnology Information (NCBI) (Bethesda, Md.) BLAST analysis of the accession number of each probe-set was done to identify each gene name. BLAST analysis identified the closest known gene existing in the database (the highest known gene at the top of the BLAST list of homologues) which then could be used to search the GeneCards database (Weizmann Institute, Rehovot, Israel). Probe-sets that did not have a known gene were labeled “EST” and their accession numbers kept as identifiers.

Human Postmortem Brain Convergence: Information about candidate genes was obtained using GeneCards, the Online Mendelian Inheritance of Man database, as well as database searches using PubMed and various combinations of keywords (gene name, schizophrenia, schizoaffective, psychosis, human, brain, postmortem, blood, lymphocytes). Postmortem convergence was deemed to occur for a gene if there were published reports of human postmortem data showing changes in expression of that gene in brains from patients with psychotic disorders (schizophrenia, schizoaffective disorder).

Human Genetic Data Convergence: To designate convergence for a particular gene, the gene had to have published positive reports from candidate gene association studies, or map within 10 cM of a microsatellite marker for which at least one published study showed evidence for genetic linkage to psychotic disorders (schizophrenia, schizoaffective disorder). The University of Southampton's sequence-based integrated map of the human genome (The Genetic Epidemiological Group, Human Genetics Division, University of Southampton (http://cedar.genetics.soton.ac.uk/public_html) was used to obtain cM locations for both genes and markers. The sex-averaged cM value was calculated and used to determine convergence to a particular marker. For markers that were not present in the Southampton database, the Marshfield database (Center for Medical Genetics, Marshfield, Wis., USA) was used with the NCBI Map Viewer web-site to evaluate linkage convergence.

Ingenuity analysis: Ingenuity Pathway Analysis 3.1 (Ingenuity Systems, Redwood City, Calif.) was used to analyze the biological roles categories of the top candidate genes resulting from the CFG analysis, as well as employed to identify genes in the datasets that are the target of existing drugs.

Convergent Functional Genomics (CFG) Analysis Scoring (FIG. 2A): Genes were given the maximum score of 2 points if changed in the human blood samples with high threshold analysis, and only 1 point if changed with low threshold. They received 1 point for each external cross-validating line of evidence (human postmortem brain data, human genetic data, animal model brain data, and animal model blood data). Genes received additional bonus points if changed in human brain and blood, as follows: 2 points if changed in the same direction, 1 point if changed in opposite direction. Genes also received additional bonus points if changed in brain and blood of the animal model, as follows: 1 point if changed in the same direction in the brain and blood, and 0.5 points if changed in opposite direction. Thus the total maximum CFG score that a candidate biomarker gene can have is 9 (2+4+2+1). Human own live subject human blood data was weighted more heavily (if it made the high threshold cut) than literature-derived human postmortem brain data, human genetic data, or the animal model data. The human blood-brain concordance data was weighted more heavily than the animal model blood-brain concordance. Other ways of weighing the scores of line of evidence may give slightly different results in terms of prioritization, if not in terms of the list of genes per se.

### Example 2

### Hallucinations Biomarkers

Using the approach for analyzing human blood gene expression data, out of over 40,000 genes and ESTs on the Affymetrix Human Genome U133 Plus 2.0 GeneChip, by using the high threshold (HT), 5 novel candidate biomarker genes were identified (Tables 3A and 5A), of which 1 had at least one line of prior independent evidence for potential involvement in mood disorders (i.e. CFG score of 3 or above). In addition to the high threshold genes, by using the low threshold, a larger list totaling 206 genes (Tables 3A and 5A), of which an additional 12 had at least two lines of prior independent evidence for potential involvement in psychotic disorders (i.e. CFG score of 3 or above) were identified. Of interest, one of the low threshold candidate biomarker genes (Phlda19) is reported to be changed in expression in the same direction, in lymphoblastoid cell lines (LCLs) from schizophrenia subjects.

Making a combined list of all the high value candidate biomarker genes identified as described above—consisting of all the high threshold genes and of the low threshold genes with at least one other external lines of evidence, and the low threshold genes with prior LCL evidence, a list of 50 top candidate biomarker genes for hallucinations, prioritized based on CFG score is identified (Table 3A).

Picking up the 5 top scoring candidate biomarkers for no hallucinations (Rhobtb3, Aldh111, Mpp3, Fn1, Spp1) and the 5 top scoring candidate biomarkers for high hallucinations (Arhgef9, S100a6, Adamts5, Pdap1, Plxnd1), a panel of 10 biomarkers for hallucinations is established that may have diagnostic and predictive value.

To test the predictive value of this panel (designated as the BioM-10 Hallucinations panel), a cohort of 31 psychotic disorders subjects was tested, containing the 23 subjects (12 no hallucinations, 11 high hallucinations) from which the candidate biomarker data was derived, as well as 8 additional subjects with hallucinations symptoms in the intermediate range (PANSS Hallucinations scores of 2 or 3). A prediction score for each subject was derived, based on the presence or absence of the 10 biomarker of the panel in their blood GeneChip data. Each of the 10 biomarkers gets a score of 1 if it is detected as Present (P) in the blood form that subject, 0.5 if it is detected as Marginally Present (M), and 0 if it is called Absent (A). The ratio of the sum of the high hallucinations biomarker scores divided by the sum of the no hallucinations biomarker scores is multiplied by 100, and provides a prediction score. If the ratio of high hallucinations biomarker genes to no hallucinations biomarker genes is 1, i.e. the two sets of genes are equally represented, the prediction score is 1×100=100. The higher this score, the higher the predicted likelihood that the subject will have high hallucinations. The predictive score with actual PANSS Hallucination scores was compared in the primary cohort of subjects with a diagnosis of psychotic disorders (n=31). A prediction score of 100 and above had a 80.0% sensitivity and a 65.0% specificity for predicting high hallucinations. A prediction score below 100 had a 91.7% sensitivity and 77.8% specificity for predicting no hallucinations (FIG. 3A and Table 4A).

Additionally, human blood gene expression analysis was conducted in a second cohort, subsequently collected, consisting of 14 subjects. The subjects in the secondary psychosis cohort had a distribution of no (n=6), intermediate (n=4) and high (n=4) hallucinations scores. The second psychosis cohort was used as a replication cohort, to verify the predictive power of the mood state biomarker panel identified by analysis of data from the primary psychosis cohort.

In the second psychosis cohort (n=14), a prediction score of 100 and above had a 75.0% sensitivity and a 55.6% specificity for predicting high hallucinations. A prediction score below 100 had a 66.7% sensitivity and 71.4% specificity for predicting no hallucinations (FIG. 3B and Table 4A).

### Example 3

### Delusions Biomarkers

Using an approach for analyzing human blood gene expression data, out of over 40,000 genes and ESTs on the Affymetrix Human Genome U133 Plus 2.0 GeneChip, by using the high threshold (HT), about 25 novel candidate biomarker genes (Tables 3B and 5B) were identified, of which 13 had at least one line of prior independent evidence for potential involvement in mood disorders (i.e. CFG score of 3 or above). In addition to the high threshold genes, by using the low threshold, a larger list totaling about 395 genes (Tables 3A and 5A) were identified, of which an additional 36 had at least two lines of prior independent evidence for potential involvement in psychotic disorders (i.e. CFG score of 3 or above). Of interest, two of the high threshold candidate biomarker genes (Egr1 and Tob2) and two of the low threshold candidate biomarker genes (Nrg1 and Gpm6b) are reported to be changed in expression in the same direction, in lymphoblastoid cell lines (LCLs) from schizophrenia subjects.

Making a combined list of all the high value candidate biomarker genes identified as described above—consisting of all the high threshold genes and of the low threshold genes with at least one other external lines of evidence, including the low threshold genes with prior LCL evidence, a list of 99 top candidate biomarker genes for delusions were identified, prioritized based on CFG score (Table 3B).

Picking up the 5 top scoring candidate biomarkers for no delusions (Drd2, ApoE, Scamp1, Idh1, Nab1)) and the 5 top scoring candidate biomarkers for high delusions (Nrg1, Egr1, Dctn1, Pllp, Pvalb), a panel of 10 biomarkers for delusions is established that may have diagnostic and predictive value.

To test the predictive value of a panel (designated as BioM-10 Delusions panel), a cohort of 31 psychotic disorders subjects, containing the 22 subjects (9 no delusions, 13 high delusions) from which the candidate biomarker data was derived, as well as 9 additional subjects with delusions symptoms in the intermediate range (PANSS Delusions scores of 2 or 3) was analyzed. A prediction score for each subject was derived, based on the presence or absence of the 10 biomarkers of the panel in their blood GeneChip data. Each of the 10 biomarkers gets a score of 1 if it is detected as Present (P) in the blood form that subject, 0.5 if it is detected as Marginally Present (M), and 0 if it is called Absent (A). The ratio of the sum of the high delusions biomarker scores divided by the sum of the no delusions biomarker scores is multiplied by 100, and provides a prediction score. If the ratio of high delusions biomarker genes to no delusions biomarker genes is 1, i.e. the two sets of genes are equally represented, the prediction score is 1×100=100. The higher this score, the higher the predicted likelihood that the subject will have high delusions. The predictive score was compared with actual PANSS Delusions scores in the primary cohort of subjects with a diagnosis of psychotic disorders (n=31). A prediction score of 100 and above had a 100% sensitivity and a 55.6% specificity for predicting high delusions. A prediction score below 100 had a 88.9% sensitivity and 90.9% specificity for predicting no delusions (FIG. 4A and Table 4B).

Additionally, human blood gene expression analysis was conducted in a second cohort, subsequently collected, consisting of 14 subjects. The subjects in the secondary psychosis cohort had a distribution of no (n=6), intermediate (n=2) and high (n=6) delusions scores. The second psychosis cohort was used as a replication cohort, to verify the predictive power of the mood state biomarker panel identified by analysis of data from the primary psychosis cohort.

In the second psychosis cohort (n=14), a prediction score of 100 and above had only a 50.0% sensitivity and a 37.5% specificity for predicting high delusions. A prediction score below 100 had a 33.3% sensitivity and 50.0% specificity for predicting no delusions (FIG. 4B and Table 4B).

### Example 4

### Clinical Applications

A sample, such as, 5-10 ml of blood is obtained from a patient suspected of having a psychotic disorder. RNA is isolated from the blood using standard protocols, for example with PAXgene blood RNA extraction kit (PreAnalytiX, a QIAGEN/BD company), followed by GLOBINclear™—Human or GLOBINclear™—Mouse/Rat (Ambion/Applied Biosystems Inc., Austin, Tex.) to remove the globin mRNA. Isolated RNA is labeled using any suitable detectable label if necessary for the gene expression analysis.

The labeled RNA is then quantified for the presence of one or more of the biomarkers disclosed herein. For example, gene expression analysis is performed using a panel of about 10 biomarkers (e.g., BioM 10 panel) for delusions and hallucinations (20 markers total) by any standard technique, for example microarray analysis or quantitative PCR or an equivalent thereof. The gene expression levels are analyzed and the fold changes (either increased, decreased, no change or absent or present) are determined and a score is established

Applications of biomarkers for psychosis: There are no reliable clinical laboratory blood tests for psychosis. Given the complex nature of psychosis, the current reliance on patient self-report of symptoms and the clinician's impression on interview of patient is a rate limiting step in delivering the best possible care with existing treatment modalities, as well as in developing new and improved treatment approaches, including new medications.

Biomarkers disclosed herein are used in the form of panels of biomarkers, as exemplified by a BioM-10 hallucination/delusion panel, for clinical laboratory tests for psychosis. Such tests can be: 1) at an mRNA level, quantitation of gene expression through polymerase chain reaction, 2) at a protein level, quantitation of protein levels through immunological approaches such as enzyme-linked immunosorbent assays (ELISA).

In conjunction with other clinical information, biomarker testing of blood and other fluids (CSF, urine) may play an important part of personalizing treatment to increase effectiveness and avoid adverse reactions—personalized medicine in psychiatry.

Biomarker-based tests for psychosis help: 1) Diagnosis, early intervention and prevention efforts; 2) Prognosis and monitoring response to various treatments; 3) New neuropsychiatric drug development efforts by pharmaceutical companies, at both a pre-clinical and clinical (Phase I, II and III) stages of the process; 4) Identifying vulnerability to psychosis for people in high stress occupations.

Example 4A: Diagnosis, early intervention and prevention efforts. A patient with no previous history of psychosis presents to a primary care doctor or internist complaining of non-specific symptoms. Such symptoms are reported in conditions such as stress after a job loss, bereavement, mononucleosis, fibromyalgia, and postpartum in the general population, as well as Gulf War syndrome in veterans. A panel of psychosis biomarkers can substantiate that the patient is showing objective changes in the blood consistent with a psychosis state. This will direct treatment towards a particular psychotic state.

Example 4B: Clinical diagnosis of a young patient. A young patient (child, adolescent, young adult) with no previous history of psychosis, but coming from a family where one or more blood relatives suffer from psychosis may be monitored with regular lab tests by their primary care doctor/pediatrician using panels of psychosis biomarkers. These tests may detect early on a change towards delusion or towards hallucination. This indicates and substantiates the need for initiation of a particular mode of treatment. This early intervention may be helpful to prevent full-blown illness and hospitalizations, with their attendant negative medical and social consequences. The decision to start medications in children and adolescents is particularly difficult without objective proof, due to the potential side-effects of medications in that age group (agitation, weigh-gain, sexual side-effects).

Example 4C: Monitoring psychosis biomarkers over an extended period. Many patients with psychosis may present initially with a depressive episode to their primary care doctor or psychiatrist. Monitoring psychosis biomarkers over time may also help to differentiate different forms of illness, e.g., depression vs. bipolar disorder (manic-depression), hallucination and others. This distinction is helpful because the first-line treatments for various psychiatric disorders are different. By seeing a change in biomarker profile towards a particular disease state before full blown illness and clinical symptoms, an appropriate addition or change to a medication can be implemented, preventing clinical decompensation, suffering and socio-economic loss (employment, relationships).

Example 4D: Prognosis and monitoring response to various treatments. It takes up to 6-8 weeks to see if a medication truly works. By doing a baseline biomarker panel test, and then a repeat test early one in treatment (after 1 week, for example), there would be an early objective indication if a medication is starting to work or not, and if a switch to another medication is indicated. This would save time and avoid needles suffering for patients, with the attendant socio-economic losses.

Example 4E: Detecting loss of efficacy of an existing treatment. When a patient has been stable for a while on a medication for psychosis, regular biomarker testing may detect early loss of efficacy of the medication or recurrence of the illness, which would indicate the dose needs to be increased, medication changed, or another medication added, to prevent full blown clinical symptoms.

Example 4F: Determining adequacy of treatment plan. Objective monitoring with blood biomarker panels of the effect of less reliable or evidence-based interventions: psychotherapy, lifestyle changes, diet and exercise programs for improving mental health. This will show whether the particular intervention works, is sufficient, or medications may need to be added to the regimen.

### Example 5

### New Neuropsychiatric Drug Development

Early-stage pre-clinical work and clinical trials of new neuropsychiatric medications for treating psychosis may benefit from biomarker monitoring to help make a decision early on whether the compound is working. This will speed up the drug-development process and avoid unnecessary costs. Depending on the expression profile of the biomarkers, the results of clinical trials may be obtained earlier than usual.

In later-stage large clinical trials, a new compound being tested may show an overall statistically non-significant positive effect, despite working well in a sub-group of people in the study. Biomarker testing may provide an objective signature of the genetic and biological make-up of the responders, which can inform recruitment for subsequent validatory clinical trials with higher likelihood of success, as well as inform which patients should be getting the medication, once it is FDA approved and on the market.

TABLE 1
Demographics: (a) individual (b) aggregate
Diagnosis established by DIGS comprehensive structured clinical interview. SZ—schizophrenia,
SZA—schizoaffective disorder. SubPD—substance induced psychosis. Psychosis score at time of blood
draw, on a scale 1 (no symptoms) to 7 (severe symptoms).
(a) Individual demographic data

| | | | | | | |
|---|---|---|---|---|---|---|
| Subject ID | Diagnosis | Age | Gender(M/F) | Race/Ethnicity | P1 Delusions (1-7) | P3 Hallucinations (1-7) |

Primary Psychosis Cohort (n = 31)

| | | | | | | |
|---|---|---|---|---|---|---|
| phchp003v1 | SZ | 50 | Male | African American | 1 | 3 |
| phchp004v1 | SZA | 55 | Male | African American | 3 | 1 |
| phchp005v1 | SZA | 45 | Male | Caucasian | 1 | 1 |
| phchp006v1 | SZA | 52 | Male | African American | 3 | 1 |
| phchp008v1 | SZ | 47 | Male | African American | 1 | 4 |
| phchp009v1 | SZ | 55 | Male | African American | 4 | 3 |
| phchp010v1 | SZA | 45 | Male | Caucasian | 2 | 2 |
| phchp012v1 | SZA | 55 | Male | Caucasian | 3 | 3 |
| phchp013v1 | SZA | 53 | Male | African American | 4 | 3 |
| phchp014v1 | SubPD | 55 | Male | African American | 2 | 3 |
| phchp015v1 | SubPD | 48 | Male | African American | 1 | 1 |
| phchp016v1 | SZ | 54 | Male | African American | 5 | 5 |
| phchp018v1 | SZA | 54 | Female | Caucasian | 6 | 4 |
| phchp019v1 | SubPD | 50 | Male | African-American | 3 | 2 |
| phchp021v1 | SZA | 48 | Male | Hispanic | 5 | 5 |
| phchp022v1 | SZ | 48 | Male | Caucasian | 2 | 1 |
| phchp024v1 | SZA | 49 | Male | African American | 2 | 4 |
| phchp025v1 | SZ | 42 | Male | Caucasian | 5 | 5 |
| phchp026v1 | SZA | 49 | Male | African-American | 4 | 4 |
| phchp033v1 | SZA | 48 | Male | Caucasian | 4 | 5 |
| phchp038v1 | SZA | 58 | Male | African-American | 1 | 1 |
| phchp040v1 | SZA | 50 | Male | Caucasian | 6 | 1 |
| phchp041v1 | SZ | 62 | Male | African-American | 5 | 5 |
| phchp042v1 | SZA | 43 | Male | Caucasian | 4 | 2 |
| phchp046v1 | SZA | 45 | Male | Caucasian | 1 | 1 |
| phchp047v1 | SZA | 57 | Male | African American | 4 | 5 |
| phchp048v1 | SZA | 56 | Male | African American | 1 | 1 |
| phchp049v1 | SZA | 46 | Male | Caucasian | 1 | 1 |
| phchp057v1 | SZA | 47 | Male | Caucasian | 1 | 1 |
| phchp061v1 | SZ | 49 | Male | Caucasian | 4 | 1 |
| phchp062v1 | SZ | 56 | Male | Caucasian | 3 | 4 |

Secondary Psychosis Cohort (n = 14)

| | | | | | | |
|---|---|---|---|---|---|---|
| phchp003v2 | SZ | 50 | Male | African American | 4 | 3 |
| phchp005v2 | SZA | 45 | Male | Caucasian | 2 | 2 |
| phchp006v2 | SZA | 52 | Male | African American | 1 | 1 |
| phchp010v3 | SZA | 45 | Male | Caucasian | 1 | 1 |
| phchp012v2 | SZA | 55 | Male | Caucasian | 4 | 5 |
| phchp013v3 | SZA | 54 | Male | African American | 4 | 5 |
| phchp016v3 | SZ | 54 | Male | African American | 4 | 4 |
| phchp017v2 | SZA | 53 | Male | African American | 1 | 1 |
| phcp021v3 | SZA | 49 | Male | Hispanic | 4 | 5 |
| phchp022v2 | SZ | 48 | Male | Caucasian | 1 | 1 |
| phchp026v3 | SZA | 49 | Male | African-American | 1 | 1 |
| phchp038v3 | SZA | 59 | Male | African-American | 1 | 1 |
| phchp040v2 | SZA | 50 | Male | Caucasian | 5 | 2 |
| phchp042v2 | SZA | 43 | Male | Caucasian | 2 | 3 |

TABLE 2
High threshold and low threshold analysis in primary psychosis
cohort.

| | Hallucinations Subjects (n = 31) |
|---|---|
| | 12 No Hallucinations and 11 High |
| Hallucinations Analysis | Hallucinations |
| High Threshold Candidate Biomarker | 9/12 No Hallucinations vs 9/11 |
| Genes (changed in greater than or | High Hallucinations |
| equal to 75% subjects; i.e. at least | A/P and P/A analysis |
| 3-fold enrichment) | |
| Low Threshold Candidate Biomarker | 8/12 No Hallucinations vs 7/11 |
| Genes (changed in greater than or | High Hallucinations |
| equal to 60% subjects; i.e. at least | A/P and P/A analysis |
| 1.5-fold enrichment) | |
| | Delusions Subjects (n = 31) |
| | 9 No Delusions and 13 High |
| Delusion Analysis | Delusions |
| High Threshold Candidate Biomarker | 7/9 No Delusions vs 10/13 High |
| Genes (changed in greater than or | Delusions |
| equal to 75% subjects; i.e. at least | A/P and P/A analysis |
| 3-fold enrichment) | |
| Low Threshold Candidate Biomarker | 6/9 No Delusions vs 8/13 High |
| Genes (changed in greater than or | Delusions |
| equal to 60% subjects; i.e. at least | A/P and P/A analysis |
| 1.5-fold enrichment) | |

Genes are considered candidate biomarkers for high psychosis if they are called by the Affymetrix MAS5 software as Absent (A) in the blood of no psychosis subjects and detected as Present (P) in the blood of high psychosis subjects. Conversely, genes are considered candidate biomarkers for no psychosis if they are detected as Present (P) in no psychosis subjects and Absent (A) in high psychosis subjects.

TABLE 3A
Top candidate biomarker genes for hallucinations (n = 50) prioritized by CFG
score for multiple independent lines of evidence.

| | | | | Human Brain | |
|---|---|---|---|---|---|
| | | | | and Blood | |
| | Affymetrix | | Human | Concordance/ | |
| Gene Symbol/ | Probeset ID/ | Human Blood | Postmortem | Co- | CFG |
| Name | Gene ID | Hallucinations | Brain | Directionality | Score |
| *Rhobtb3 | 216048_s_at | D | Down | Yes/Yes | 5 |
| Rho-related BTB | 22836 | | | | |
| domain | | | | | |
| containing 3 | | | | | |
| *Aldh1l1 | 205208_at | D | Down | Yes/Yes | 4 |
| aldehyde | 10840 | | | | |
| dehydrogenase 1 | | | | | |
| family, member | | | | | |
| L1 | | | | | |
| *Arhgef9 | 203264_s_at | I | Down | Yes/No | 4 |
| Cdc42 guanine | 23229 | | | | |
| nucleotide | | | | | |
| exchange factor | | | | | |
| (GEF) 9 | | | | | |
| *Fn1 | 1558199_at | D (HT) | | | 4 |
| fibronectin 1 | 2335 | | | | |
| *Mpp3 | 206186_at | D | Up | Yes/No | 4 |
| membrane | 4356 | | | | |
| protein, | | | | | |
| palmitoylated 3 | | | | | |
| (MAGUK p55 | | | | | |
| subfamily | | | | | |
| member 3) | | | | | |
| *S100a6 | 228923_at | I | Down | Yes/No | 4 |
| S100 calcium | 6277 | | | | |
| binding protein | | | | | |
| A6 (calcyclin) | | | | | |
| *Spp1 | 209875_s_at | D | | | 3 |
| secreted | 6696 | | | | |
| phosphoprotein 1 | | | | | |
| (osteopontin, | | | | | |
| bone sialoprotein | | | | | |
| I, early T- | | | | | |
| lymphocyte | | | | | |
| activation 1) | | | | | |
| *Adamts5 | 229357_at | I | | | 3 |
| ADAM | 11096 | | | | |
| metallopeptidase | | | | | |
| with | | | | | |
| thrombospondin | | | | | |
| type 1 motif, 5 | | | | | |
| (aggrecanase-2) | | | | | |
| Add2 | 206807_s_at | D | | | 3 |
| adducin 2 (beta) | 119 | | | | |
| Eif4g3 | 1554309_at | D | | | 3 |
| eukaryotic | 8672 | | | | |
| translation | | | | | |
| initiation factor 4 | | | | | |
| gamma, 3 | | | | | |
| *Pdap1 | 217624_at | I | | | 3 |
| PDGFA | 11333 | | | | |
| associated | | | | | |
| protein 1 | | | | | |
| Ptpla | 219654_at | D | | | 3 |
| protein tyrosine | 9200 | | | | |
| phosphatase-like | | | | | |
| (proline instead of | | | | | |
| catalytic | | | | | |
| arginine), | | | | | |
| member A | | | | | |
| Rhoj | 235131_at | D | | | 3 |
| ras homolog | 57381 | | | | |
| gene family, | | | | | |
| member J | | | | | |
| *Plxnd1 | 212235_at | I | | | 2 |
| Plexin D1 | 23129 | | | | |
| Pacs2 | 1555824_a_at | I | | | 2 |
| phosphofurin | 23241 | | | | |
| acidic cluster | | | | | |
| sorting protein 2 | | | | | |
| USP53 | 216775_at | I | | | 2 |
| ubiquitin specific | 54532 | | | | |
| peptidase 53 | | | | | |
| Zcchc12 | 228715_at | D | | | 2 |
| zinc finger, | 170261 | | | | |
| CCHC domain | | | | | |
| containing 12 | | | | | |
| Hemk1 | 218621_at | D | | | 2 |
| HemK | 51409 | | | | |
| methyltransferase | | | | | |
| family member 1 | | | | | |
| Map6d1 | 221713_s_at | I | | | 2 |
| MAP6 domain | 79929 | | | | |
| containing 1 | | | | | |
| Wdr68 | 209592_s_at | D | | | 2 |
| WD repeat | 10238 | | | | |
| domain 68 | | | | | |
| Nefh | 33767_at | I | | | 2 |
| neurofilament, | 4744 | | | | |
| heavy | | | | | |
| polypeptide | | | | | |
| 200 kDa | | | | | |
| Mcf2 | 217004_s_at | I | | | 2 |
| mcf.2 | 4168 | | | | |
| transforming | | | | | |
| sequence | | | | | |
| Adarb1 | 207999_s_at | I | | | 2 |
| adenosine | 104 | | | | |
| deaminase, RNA- | | | | | |
| specific, B1 | | | | | |
| Ube2i | 213536_s_at | I | | | 2 |
| ubiquitin- | 7329 | | | | |
| conjugating | | | | | |
| enzyme E2I | | | | | |
| Cntnap2 | 219301_s_at | D | | | 2 |
| contactin | 26047 | | | | |
| associated | | | | | |
| protein-like 2 | | | | | |
| Loc400120 | 241672_at | I | | | 2 |
| hypothetical | 400120 | | | | |
| LOC400120 | | | | | |
| Znf24 | 203248_at | I | | | 2 |
| zinc finger protein | 7572 | | | | |
| 24 | | | | | |
| Actr3b | 1555487_a_at | D | | | 2 |
| ARP3 actin- | 57180 | | | | |
| related protein 3 | | | | | |
| homolog B | | | | | |
| (yeast) | | | | | |
| Afap1 | 203563_at | D | | | 2 |
| actin filament | 60312 | | | | |
| associated | | | | | |
| protein 1 | | | | | |
| Slc6a13 | 237058_x_at | I | | | 2 |
| solute carrier | 6540 | | | | |
| family 6 | | | | | |
| (neurotransmitter | | | | | |
| transporter, | | | | | |
| GABA), member | | | | | |
| 13 | | | | | |
| Fbxo2 | 219305_x_at | I | | | 2 |
| F-box only | 26232 | | | | |
| protein 2 | | | | | |
| Gnai1 | 227692_at | I | | | 2 |
| guanine | 2770 | | | | |
| nucleotide | | | | | |
| binding protein, | | | | | |
| alpha inhibiting 1 | | | | | |
| Wdr33 | 223146_at | I | | | 2 |
| WD repeat | 55339 | | | | |
| domain 33 | | | | | |
| Otud4 | 220669_at | D | | | 2 |
| OTU domain | 54726 | | | | |
| containing 4 | | | | | |
| Rnf182 | 230720_at | D | | | 2 |
| ring finger protein | 221687 | | | | |
| 182 | | | | | |
| SYNJ2 | 216180_s_at | D | | | 2 |
| synaptojanin 2 | 8871 | | | | |
| Neo1 | 204321_at | I | | | 2 |
| neogenin | 4756 | | | | |
| homolog 1 | | | | | |
| (chicken) | | | | | |
| RANGAP1 | 212127_at | I | | | 2 |
| Ran GTPase | 5905 | | | | |
| activating protein 1 | | | | | |
| Stk32c | 227634_at | I | | | 2 |
| serine/threonine | 282974 | | | | |
| kinase 32C | | | | | |
| Adrbk2 | 204183_s_at | I | | | 2 |
| adrenergic | 157 | | | | |
| receptor kinase, | | | | | |
| beta 2 | | | | | |
| Camkv | 219365_s_at | I | | | 2 |
| CaM kinase-like | 79012 | | | | |
| vesicle- | | | | | |
| associated | | | | | |
| Atp2c1 | 209935_at | I | | | 2 |
| ATPase, Ca++- | 27032 | | | | |
| sequestering | | | | | |
| MGST1 | 239001_at | I | | | 2 |
| microsomal | 4257 | | | | |
| glutathione S- | | | | | |
| transferase 1 | | | | | |
| PTK2 | 241453_at | D | | | 2 |
| PTK2 protein | 5747 | | | | |
| tyrosine kinase 2 | | | | | |
| Tmtc1 | 224397_s_at | I | | | 2 |
| transmembrane | 83857 | | | | |
| and | | | | | |
| tetratricopeptide | | | | | |
| repeat containing 1 | | | | | |
| PLS3 | 201215_at | D (HT) | | | 2 |
| plastin 3 (T | 5358 | | | | |
| isoform) | | | | | |
| FIZ1 | 226967_at | I (HT) | | | 2 |
| FLT3-interacting | 84922 | | | | |
| zinc finger 1 | | | | | |
| MGC2752 | 1568864_at | I (HT) | | | 2 |
| Hypothetical | 65996 | | | | |
| protein MGC2752 | | | | | |
| PSD4 | 215923_s_at | I (HT) | | | 2 |
| pleckstrin and | 23550 | | | | |
| Sec7 domain | | | | | |
| containing 4 | | | | | |
| Phlda1 | 225842_at | I | | | 2 |
| pleckstrin | 22822 | Up | | | |
| homology-like | | Schizophrenia | | | |
| domain, family A, | | lymphocytes⁹ | | | |
| member 1 | | | | | |

Top candidate biomarker genes for hallucinations.
For human blood data:
I - increased in high hallucinations state;
D - decreased in high hallucinations state/increased in no hallucinations state.
For postmortem brain data:
Up - increased;
Down - decreased in expression;
PCP - phencyclidine,
CLZ - clozapine;
(HT) High threshold.
Highlighted with an asterisk - BioM 10 markers.

TABLE 3B
Top candidate biomarker genes for delusions (n = 99) prioritized by CFG
score for multiple independent lines of evidence.

| | | | | Human Brain | |
|---|---|---|---|---|---|
| | | | | and Blood | |
| | Affymetrix | | Human | Concordance/ | |
| | Probeset ID/ | Human Blood | Postmortem | Co- | CFG |
| Gene Symbol/Name | Gene ID | Delusions | Brain | Directionality | Score |
| *Drd2 | 216938_x_at | D | Down | Yes/Yes | 6 |
| dopamine receptor 2 | 1813 | | | | |
| *Egr1 | 201693_s_at | I (HT) | Down | Yes/No | 6 |
| early growth | 1958 | | | | |
| response 1 | | | | | |
| *Apoe | 212884_x_at | D | Down | Yes/Yes | 5 |
| Apolipoprotein E | 348 | | | | |
| *Dctn1 | 211780_x_at | I (HT) | Down | Yes/No | 5 |
| dynactin 1 (p150, | 1639 | | | | |
| glued homolog, | | | | | |
| Drosophila) | | | | | |
| *Idh1 | 242001_at | D | Down | Yes/Yes | 5 |
| Isocitrate | 3417 | | | | |
| dehydrogenase 1 | | | | | |
| (NADP+), soluble | | | | | |
| *Nab1 | 208047_s_at | D | | | 5 |
| NGFI-A binding | 4664 | | | | |
| protein 1 (EGR1 | | | | | |
| binding protein 1) | | | | | |
| *Nrg1 | 208241_at | I | Up | Yes/Yes | 5 |
| neuregulin 1 | 3084 | | | | |
| *Scamp1 | 1570210_x_at | D | Down | Yes/Yes | 5 |
| secretory carrier | 9522 | | | | |
| membrane protein 1 | | | | | |
| *Aldh1l1 | 205208_at | D | Down | Yes/Yes | 4 |
| aldehyde | 10840 | | | | |
| dehydrogenase 1 | | | | | |
| family, member L1 | | | | | |
| *Gpm6b | 209168_at | D | Up | Yes/No | 4 |
| Glycoprotein M6B | 2824 | | | | |
| *Ncoa2 | 205732_s_at | D | | | 4 |
| Nuclear receptor | 10499 | | | | |
| coactivator 2 | | | | | |
| *Pllp | 204519_s_at | I | Down | Yes/No | 4 |
| plasma membrane | 51090 | | | | |
| proteolipid | | | | | |
| (plasmolipin) | | | | | |
| *Pvalb | 205336_at | I | Down | Yes/No | 4 |
| parvalbumin | 5816 | | | | |
| Nmt1 | 201159_s_at | I | Up | Yes/Yes | 4 |
| N- | 4836 | | | | |
| myristoyltransferase 1 | | | | | |
| Pctk1 | 208823_s_at | I | Down | Yes/No | 4 |
| PCTAIRE-motif | 5127 | | | | |
| protein kinase 1 | | | | | |
| Stxbp6 | 220995_at | D | | | 3.5 |
| syntaxin binding | 29091 | | | | |
| protein 6 (amisyn) | | | | | |
| Human Brain | | | | | |
| Adam9 | 1570042_a_at | D | Up | Yes/No | 3 |
| ADAM | 8754 | | | | |
| metallopeptidase | | | | | |
| domain 9 (meltrin | | | | | |
| gamma) | | | | | |
| Adamts5 | 229357_at | I | | | 3 |
| ADAM | 11096 | | | | |
| metallopeptidase with | | | | | |
| thrombospondin type | | | | | |
| 1 motif, 5 | | | | | |
| (aggrecanase-2) | | | | | |
| Add2 | 206807_s_at | D | | | 3 |
| adducin 2 (beta) | 119 | | | | |
| Bin3 | 1557582_at | I (HT) | | | 3 |
| bridging integrator 3 | 55909 | | | | |
| Cd84 | 211192_s_at | I | | | 3 |
| CD84 molecule | 8832 | | | | |
| Clasp1 | 240757_at | D | | | 3 |
| CLIP associating | 23332 | | | | |
| protein 1 | | | | | |
| Dnm2 | 1555895_at | I | | | 3 |
| dynamin 2 | 1785 | | | | |
| Fn1 | 1558199_at | D | | | 3 |
| fibronectin 1 | 2335 | | | | |
| Gas2l1 | 209729_at | I (HT) | | | 3 |
| growth arrest-specific | 10634 | | | | |
| 2 like 1 | | | | | |
| Gpm6a | 209470_s_at | D (HT) | | | 3 |
| glycoprotein m6a | 2823 | | | | |
| Hbp1 | 236645_at | I | | | 3 |
| HMG-box | 26959 | | | | |
| transcription factor 1 | | | | | |
| Herc3 | 1554290_at | D | | | 3 |
| hect domain and RLD 3 | 8916 | | | | |
| Hfe | 211332_x_at | I (HT) | | | 3 |
| hemochromatosis | 3077 | | | | |
| Kif5c | 203129_s_at | I | | | 3 |
| kinesin family | 3800 | | | | |
| member 5C | | | | | |
| Ltbp3 | 227308_x_at | I | | | 3 |
| latent transforming | 4054 | | | | |
| growth factor beta | | | | | |
| binding protein 3 | | | | | |
| Mat2b | 229284_at | D | | | 3 |
| methionine | 27430 | | | | |
| adenosyltransferase | | | | | |
| II, beta | | | | | |
| Mfrp | 224286_at | I (HT) | | | 3 |
| membrane frizzled- | 83552 | | | | |
| related protein | | | | | |
| Mgea5 | 235868_at | D (HT) | | | 3 |
| Meningioma | 10724 | | | | |
| expressed antigen 5 | | | | | |
| (hyaluronidase) | | | | | |
| Mpp5 | 219321_at | D | | | 3 |
| membrane protein, | 64398 | | | | |
| palmitoylated 5 | | | | | |
| (MAGUK p55 | | | | | |
| subfamily member 5) | | | | | |
| Mrpl39 | 236910_at | D (HT) | | | 3 |
| Mitochondrial | 54148 | | | | |
| ribosomal protein L39 | | | | | |
| Ppap2a | 209147_s_at | I | | | 3 |
| phosphatidic acid | 8611 | | | | |
| phosphatase 2a | | | | | |
| Prickle1 | 232811_x_at | D | | | 3 |
| prickle like 1 | 144165 | | | | |
| (Drosophila) | | | | | |
| Rcc1 | 206499_s_at | I (HT) | | | 3 |
| regulator of | 1104 | | | | |
| chromosome | | | | | |
| condensation 1 | | | | | |
| Sh3bp4 | 232691_at | D | | | 3 |
| SH3-domain binding | 23677 | | | | |
| protein 4 | | | | | |
| Sparc | 212667_at | I | | | 3 |
| secreted protein, | 6678 | | | | |
| acidic, cysteine-rich | | | | | |
| (osteonectin) | | | | | |
| Tmem106b | 233666_at | D | Up¹⁰ | Yes/No | 3 |
| transmembrane | 54664 | | | | |
| protein 106B | | | | | |
| Tp5s3i11 | 203421_at | I (HT) | | | 3 |
| tumor protein p53 | 9537 | | | | |
| inducible protein 11 | | | | | |
| Tpp1 | 214195_at | I (HT) | | | 3 |
| tripeptidyl peptidase I | 1200 | | | | |
| Vangl1 | 229134_at | I | | | 3 |
| vang-like 1 (van | 81839 | | | | |
| gogh, Drosophila) | | | | | |
| Yaf2 | 206238_s_at | D | | | 3 |
| YY1 associated factor 2 | 10138 | | | | |
| Plxnd1 | 212235_at | I | | | 2 |
| Plexin D1 | 23129 | | | | |
| Actr3b | 1555487_a_at | D | | | 2 |
| ARP3 actin-related | 57180 | | | | |
| protein 3 homolog B | | | | | |
| (yeast) | | | | | |
| Adarb1 | 207999_s_at | I | | | 2 |
| adenosine | 104 | | | | |
| deaminase, RNA- | | | | | |
| specific, B1 | | | | | |
| Angel2 | 217630_at | D | | | 2 |
| angel homolog 2 | 90806 | | | | |
| (Drosophila) | | | | | |
| Arid1b | 1566989_at | D | | | 2 |
| AT rich interactive | 57492 | | | | |
| domain 1B (SWI1- | | | | | |
| like) | | | | | |
| Atp2c1 | 209935_at | I | | | 2 |
| ATPase, Ca++- | 27032 | | | | |
| sequestering | | | | | |
| B3galt2 | 210121_at | D | | | 2 |
| UDP-Gal:betaGlcNAc | 8707 | | | | |
| beta 1,3- | | | | | |
| galactosyltransferase, | | | | | |
| polypeptide 2 | | | | | |
| BCORL1 | 234711_s_at | D (HT) | | | 2 |
| BCL6 co-repressor- | 63035 | | | | |
| like 1 | | | | | |
| Bsdc1 | 1559971_at | D | | | 2 |
| BSD domain | 55108 | | | | |
| containing 1 | | | | | |
| C10orf4 | 238596_at | D | | | 2 |
| chromosome 10 open | 118924 | | | | |
| reading frame 4 | | | | | |
| C19orf55 | 242640_at | I (HT) | | | 2 |
| chromosome 19 open | 148137 | | | | |
| reading frame 55 | | | | | |
| C1orf96 | 1553697_at | D | | | 2 |
| chromosome 1 open | 126731 | | | | |
| reading frame 96 | | | | | |
| Calml4 | 1566150_at | D | | | 2 |
| calmodulin-like 4 | 91860 | | | | |
| Camkv | 219365_s_at | I | | | 2 |
| CaM kinase-like | 79012 | | | | |
| vesicle-associated | | | | | |
| Camsap1l1 | 212763_at | D | | | 2 |
| calmodulin regulated | 23271 | | | | |
| spectrin-associated | | | | | |
| protein 1-like 1 | | | | | |
| Dock9 | 232874_at | I | | | 2 |
| Dedicator of | 23348 | | | | |
| cytokinesis 9 | | | | | |
| FAM137A | 236214_at | D (HT) | | | 2 |
| family with sequence | 84691 | | | | |
| similarity 137, | | | | | |
| member A | | | | | |
| Fam70a | 219895_at | D | | | 2 |
| family with sequence | 55026 | | | | |
| similarity 70, member A | | | | | |
| Fosl2 | 218881_s_at | I | | | 2 |
| FOS-like antigen 2 | 2355 | | | | |
| Gp9 | 206883_x_at | I | | | 2 |
| glycoprotein 9 | 2815 | | | | |
| (platelet) | | | | | |
| GPR84 | 223767_at | I (HT) | | | 2 |
| G protein-coupled | 53831 | | | | |
| receptor 84 | | | | | |
| Hbe1 | 205919_at | D | | | 2 |
| hemoglobin, epsilon 1 | 3046 | | | | |
| HIPK3 | 207764_s_at | I | | | 2 |
| homeodomain | 10114 | | | | |
| interacting protein | | | | | |
| kinase 3 | | | | | |
| INTS7 | 222250_s_at | D | | | 2 |
| integrator complex | 25896 | | | | |
| subunit 8 | | | | | |
| IQCH | 1569611_a_at | D (HT) | | | 2 |
| IQ motif containing H | 64799 | | | | |
| KIR2DS2 | 211532_x_at | I (HT) | | | 2 |
| killer cell | 3807 | | | | |
| immunoglobulin-like | | | | | |
| receptor, two | | | | | |
| domains, short | | | | | |
| cytoplasmic tail, 2 | | | | | |
| KLK2 | 1555545_at | I (HT) | | | 2 |
| kallikrein-related | 3817 | | | | |
| peptidase 2 | | | | | |
| LOC90835 | 231300_at | I (HT) | | | 2 |
| hypothetical protein | 90835 | | | | |
| LOC90835 | | | | | |
| Lrch1 | 235012_at | D | | | 2 |
| leucine-rich repeats | 23143 | | | | |
| and calponin | | | | | |
| homology (CH) | | | | | |
| domain containing 1 | | | | | |
| Map4k5 | 211081_s_at | D | | | 2 |
| mitogen-activated | 11183 | | | | |
| protein kinase kinase | | | | | |
| kinase kinase 5 | | | | | |
| March7 | 1557704_a_at | D | | | 2 |
| membrane- | 64844 | | | | |
| associated ring finger | | | | | |
| (C3HC4) 7 | | | | | |
| MGC16075 | 1553708_at | D (HT) | | | 2 |
| hypothetical protein | 84847 | | | | |
| MGC16075 | | | | | |
| MGST1 | 239001_at | I | | | 2 |
| microsomal | 4257 | | | | |
| glutathione S- | | | | | |
| transferase 1 | | | | | |
| NAV2 | 218330_s_at | D (HT) | | | 2 |
| neuron navigator 2 | 89797 | | | | |
| Pacs2 | 1555824_a_at | I | | | 2 |
| phosphofurin acidic | 23241 | | | | |
| cluster sorting protein 2 | | | | | |
| Phf3 | 217953_at | D | | | 2 |
| PHD finger protein 3 | 23469 | | | | |
| PLS3 | 201215_at | D (HT) | | | 2 |
| plastin 3 (T isoform) | 5358 | | | | |
| PTK2 | 241453_at | D | | | 2 |
| PTK2 protein tyrosine | 5747 | | | | |
| kinase 2 | | | | | |
| RANGAP1 | 212125_at | I | | | 2 |
| Ran GTPase | 5905 | | | | |
| activating protein 1 | | | | | |
| Slc13a4 | 233230_s_at | D | | | 2 |
| solute carrier family | 26266 | | | | |
| 13 (sodium/sulfate | | | | | |
| symporters), member 4 | | | | | |
| SNX21 | 1553961_s_at | I | | | 2 |
| sorting nexin family | 90203 | | | | |
| member 21 | | | | | |
| Stk32c | 227634_at | I | | | 2 |
| serine/threonine | 282974 | | | | |
| kinase 32C | | | | | |
| Tcf4 | 212385_at | D | | | 2 |
| transcription factor 4 | 6925 | | | | |
| THTPA | 214341_at | I (HT) | | | 2 |
| Thiamine | 79178 | | | | |
| triphosphatase | | | | | |
| Tmem158 | 213338_at | I | | | 2 |
| transmembrane | 25907 | | | | |
| protein 158 | | | | | |
| Tmem64 | 242338_at | D | | | 2 |
| transmembrane | 169200 | | | | |
| protein 64 | | | | | |
| Tmtc2 | 235775_at | I | | | 2 |
| transmembrane and | 160335 | | | | |
| tetratricopeptide | | | | | |
| repeat containing 2 | | | | | |
| Tob2 | 221496_s_at | I (HT) | | | 2 |
| transducer of ERBB2, 2 | 10766 | Up | | | |
| | | Schizophrenia | | | |
| | | lymphocytes | | | |
| Tsc22d3 | 235364_at | D | | | 2 |
| TSC22 domain | 1831 | | | | |
| family, member 3 | | | | | |
| Twf1 | 243033_at | D | | | 2 |
| twinfilin, actin-binding | 5756 | | | | |
| protein, homolog 1 | | | | | |
| (Drosophila) | | | | | |
| USP53 | 216775_at | I | | | 2 |
| ubiquitin specific | 54532 | | | | |
| peptidase 53 | | | | | |
| Zcchc12 | 228715_at | D | | | 2 |
| zinc finger, CCHC | 170261 | | | | |
| domain containing 12 | | | | | |

Top candidate biomarker genes for hallucinations.
For human blood data:
I - increased in high delusions state;
D - decreased in high delusions state/increased in no delusions state.
For postmortem brain data:
Up - increased;
Down - decreased in expression;
PCP - phencyclidine,
CLZ - clozapine;
(HT) High threshold.
Asterisk - BioM 10 markers.

TABLE 4
BioM-10 Psychosis panels sensitivity and specificity for
predicting psychosis state.

| | |
|---|---|
| Sensitivity | Specificity |

A. (Hallucinations).

| | | |
|---|---|---|
| Primary Psychosis | | |
| Cohort | | |
| Hallucinations | 80.0% | 65.0% |
| No Hallucinations | 91.7% | 77.8% |
| Secondary Psychosis | | |
| Cohort | | |
| Hallucinations | 75.0% | 55.6% |
| No Hallucinations | 66.7% | 71.4% |

B. (Delusions).

| | | |
|---|---|---|
| Primary Psychosis | | |
| Cohort | | |
| Delusions | 100.0% | 55.6% |
| No Delusions | 88.9% | 90.9% |
| Secondary Psychosis | | |
| Cohort | | |
| Delusions | 50.0% | 37.5% |
| No Delusions | 33.3% | 50.0% |

(a) Hallucinations
(B) Delusions

TABLE 5A
Complete list of candidate biomarker genes for hallucinations
(n = 211) identified using A/P analysis and CFG scoring

| | Human Blood | CFG |
|---|---|---|
| Gene Symbol/Gene Name | Hallucinations | Score |
| Rhobtb3 Rho-related BTB domain containing 3 | D | 5 |
| S100A6 S100 calcium binding protein A6 | I | 4 |
| (calcyclin) | | |
| ALDH1L1 aldehyde dehydrogenase 1 family, | D | 4 |
| member L1 | | |
| Arhgef9 Cdc42 guanine nucleotide exchange | I | 4 |
| factor (GEF) 9 | | |
| PHLDA1 pleckstrin homology-like domain, | I | 4 |
| family A, member 1 | | |
| Mpp3 membrane protein, palmitoylated 3 | D | 4 |
| (MAGUK p55 subfamily member 3) | | |
| Fn1 fibronectin 1 | D (HT) | 4 |
| Eif4g3 eukaryotic translation initiation factor 4 | D | 3 |
| gamma, 3 | | |
| Pdap1 PDGFA associated protein 1 | I | 3 |
| Add2 adducin 2 (beta) | D | 3 |
| Spp1 secreted phosphoprotein 1 | D | 3 |
| (osteopontin, bone sialoprotein I, early T- | | |
| lymphocyte activation 1) | | |
| Adamts5 ADAM metallopeptidase with | I | 3 |
| thrombospondin type 1 motif, 5 | | |
| (aggrecanase-2) | | |
| Rhoj ras homolog gene family, member J | D | 3 |
| Ptpla protein tyrosine phosphatase-like | D | 3 |
| (proline instead of catalytic arginine), member A | | |
| Plxnd1 Plexin D1 | I | 2 |
| Pacs2 phosphofurin acidic cluster sorting | I | 2 |
| protein 2 | | |
| USP53 ubiquitin specific peptidase 53 | I | 2 |
| Zcchc12 zinc finger, CCHC domain | D | 2 |
| containing 12 | | |
| Hemk1 HemK methyltransferase family | D | 2 |
| member 1 | | |
| Map6d1 MAP6 domain containing 1 | I | 2 |
| Wdr68 WD repeat domain 68 | D | 2 |
| Nefh neurofilament, heavy polypeptide | I | 2 |
| 200 kDa | | |
| Mcf2 mcf.2 transforming sequence | I | 2 |
| Adarb1 adenosine deaminase, RNA-specific, | I | 2 |
| B1 | | |
| Ube2i ubiquitin-conjugating enzyme E2I | I | 2 |
| Cntnap2 contactin associated protein-like 2 | D | 2 |
| Loc400120 hypothetical LOC400120 | I | 2 |
| Znf24 zinc finger protein 24 | I | 2 |
| Actr3b ARP3 actin-related protein 3 homolog | D | 2 |
| B (yeast) | | |
| Afap1 actin filament associated protein 1 | D | 2 |
| Slc6a13 solute carrier family 6 | I | 2 |
| (neurotransmitter transporter, GABA), | | |
| member 13 | | |
| Fbxo2 F-box only protein 2 | I | 2 |
| Gnai1 guanine nucleotide binding protein, | I | 2 |
| alpha inhibiting 1 | | |
| Wdr33 WD repeat domain 33 | I | 2 |
| Otud4 OTU domain containing 4 | D | 2 |
| Rnf182 ring finger protein 182 | D | 2 |
| SYNJ2 synaptojanin 2 | D | 2 |
| Neo1 neogenin homolog 1 (chicken) | I | 2 |
| RANGAP1 Ran GTPase activating protein 1 | I | 2 |
| Stk32c serine/threonine kinase 32C | I | 2 |
| Adrbk2 adrenergic receptor kinase, beta 2 | I | 2 |
| Camkv CaM kinase-like vesicle-associated | I | 2 |
| Atp2c1 ATPase, Ca++-sequestering | I | 2 |
| MGST1 microsomal glutathione S-transferase 1 | I | 2 |
| PTK2 PTK2 protein tyrosine kinase 2 | D | 2 |
| Tmtc1 transmembrane and tetratricopeptide | I | 2 |
| repeat containing 1 | | |
| RCC1 /// SNHG3-RCC1 regulator of | I | 2 |
| chromosome condensation 1 | | |
| TPP1 tripeptidyl peptidase I | I | 2 |
| Atxn1 Ataxin 1 | D | 2 |
| BCORL1 BCL6 co-repressor-like 1 | D | 2 |
| FAM137A family with sequence similarity 137, | D | 2 |
| member A | | |
| NAV2 neuron navigator 2 | D | 2 |
| TEX261 testis expressed sequence 261 | D | 2 |
| PLS3 plastin 3 (T isoform) | D (HT) | 2 |
| C14orf1 chromosome 14 open reading frame 1 | I | 2 |
| Egln3 EGL nine homolog 3 (C. elegans) | I | 2 |
| KLK2 kallikrein-related peptidase 2 | I | 2 |
| Loc51035 SAPK substrate protein 1 | I | 2 |
| LOC90835 hypothetical protein LOC90835 | I | 2 |
| THTPA Thiamine triphosphatase | I | 2 |
| ZFP36L1 zinc finger protein 36, C3H type-like 1 | I | 2 |
| FIZ1 FLT3-interacting zinc finger 1 | I (HT) | 2 |
| MGC2752 Hypothetical protein MGC2752 | I (HT) | 2 |
| PSD4 pleckstrin and Sec7 domain containing 4 | I (HT) | 2 |
| MICAL2 microtubule associated | D | 1 |
| monoxygenase, calponin and LIM domain | | |
| containing 2 | | |
| ABCB4 ATP-binding cassette, sub-family B | D | 1 |
| (MDR/TAP), member 4 | | |
| ALS2CR4 amyotrophic lateral sclerosis 2 | D | 1 |
| (juvenile) chromosome region, candidate 4 | | |
| ASB2 ankyrin repeat and SOCS box- | D | 1 |
| containing 2 | | |
| C1orf128 chromosome 1 open reading frame | D | 1 |
| 128 | | |
| C20orf42 chromosome 20 open reading | D | 1 |
| frame 42 | | |
| C2orf59 chromosome 2 open reading frame | D | 1 |
| 59 | | |
| C4orf18 chromosome 4 open reading frame | D | 1 |
| 18 | | |
| CACYBP calcyclin binding protein | D | 1 |
| CD276 CD276 molecule | D | 1 |
| CPA5 carboxypeptidase A5 | D | 1 |
| CYP2A6 cytochrome P450, family 2, | D | 1 |
| subfamily A, polypeptide 6 | | |
| DDX19A DEAD (Asp-Glu-Ala-As) box | D | 1 |
| polypeptide 19A | | |
| DMBT1 deleted in malignant brain tumors 1 | D | 1 |
| DNAJC11 DnaJ (Hsp40) homolog, subfamily | D | 1 |
| C, member 11 | | |
| DSP desmoplakin | D | 1 |
| Entpd4 ectonucleoside triphosphate | D | 1 |
| diphosphohydrolase 4 | | |
| ERICH1 Glutamate-rich 1 | D | 1 |
| Fgf13 fibroblast growth factor 13 | D | 1 |
| FLJ44894 similar to zinc finger protein 91 | D | 1 |
| G3BP1 | D | 1 |
| Gtdc1 glycosyltransferase-like domain | D | 1 |
| containing 1 | | |
| HCRP1 hepatocellular carcinoma-related | D | 1 |
| HCRP1 | | |
| HLA-C /// IGKC /// IGKV1-5 major | D | 1 |
| histocompatibility complex, class I, C /// | | |
| immunoglobulin kappa constant /// | | |
| immunoglobulin kappa variable 1-5 | | |
| IGHV1-69 Immunoglobulin heavy variable 1- | D | 1 |
| 69 | | |
| KLHDC8A kelch domain containing 8A | D | 1 |
| LOC145783 | D | 1 |
| LOC646677 /// LOC650674 similar to | D | 1 |
| aconitase 2, mitochondrial | | |
| LYZL6 lysozyme-like 6 | D | 1 |
| MREG melanoregulin | D | 1 |
| MYO1A myosin IA | D | 1 |
| NCKIPSD NCK interacting protein with SH3 | D | 1 |
| domain | | |
| NHEDC1 | D | 1 |
| OSGEP O-sialoglycoprotein endopeptidase | D | 1 |
| PCGF2 polycomb group ring finger 2 | D | 1 |
| PCOLCE2 procollagen C-endopeptidase | D | 1 |
| enhancer 2 | | |
| PFDN4 prefoldin subunit 4 | D | 1 |
| PIK3C2A phosphoinositide-3-kinase, class 2, | D | 1 |
| alpha polypeptide | | |
| PPP1R3B protein phosphatase 1, regulatory | D | 1 |
| (inhibitor) subunit 3B | | |
| RAB26 RAB26, member RAS oncogene | D | 1 |
| family | | |
| RPAP3 RNA polymerase II associated protein 3 | D | 1 |
| SLC28A3 solute carrier family 28 (sodium- | D | 1 |
| coupled nucleoside transporter), member 3 | | |
| ST3GAL3 ST3 beta-galactoside alpha-2,3- | D | 1 |
| sialyltransferase 3 | | |
| SYCP3 synaptonemal complex protein 3 | D | 1 |
| TAF11 TAF11 RNA polymerase II, TATA box | D | 1 |
| binding protein (TBP)-associated factor, | | |
| 28 kDa | | |
| TRIM69 tripartite motif-containing 69 | D | 1 |
| WDR73 WD repeat domain 73 | D | 1 |
| YOD1 YOD1 OTU deubiquinating enzyme 1 | D | 1 |
| homolog (S. cerevisiae) | | |
| ZNF683 zinc finger protein 683 | D | 1 |
| ADAT3 adenosine deaminase, tRNA-specific | I | 1 |
| 3, TAD3 homolog (S. cerevisiae) | | |
| Akap8l A kinase (PRKA) anchor protein 8-like | I | 1 |
| ALG10 asparagine-linked glycosylation 10 | I | 1 |
| homolog (yeast, alpha-1,2- | | |
| glucosyltransferase) | | |
| ALS2 amyotrophic lateral sclerosis 2 | I | 1 |
| (juvenile) | | |
| ANKRD5 ankyrin repeat domain 5 | I | 1 |
| ANKRD52 ankyrin repeat domain 52 | I | 1 |
| ATAD3A ATPase family, AAA domain | I | 1 |
| containing 3A | | |
| BACE1 beta-site APP-cleaving enzyme 1 | I | 1 |
| C11orf35 chromosome 11 open reading | I | 1 |
| frame 35 | | |
| C12orf51 chromosome 12 open reading | I | 1 |
| frame 51 | | |
| C17orf69 chromosome 17 open reading | I | 1 |
| frame 69 | | |
| C2orf3 chromosome 2 open reading frame 3 | I | 1 |
| C7orf16 chromosome 7 open reading frame | I | 1 |
| 16 | | |
| CCDC136 coiled-coil domain containing 136 | I | 1 |
| CCNL2 /// LOC727877 cyclin L2 /// similar to | I | 1 |
| Cyclin-L2 (Paneth cell-enhanced expression | | |
| protein) | | |
| CD6 CD6 molecule | I | 1 |
| CLCC1 Chloride channel CLIC-like 1 | I | 1 |
| CLPTM1 cleft lip and palate associated | I | 1 |
| transmembrane protein 1 | | |
| Cltc clathrin, heavy polypeptide (Hc) | I | 1 |
| COPZ2 coatomer protein complex, subunit | I | 1 |
| zeta 2 | | |
| CREB3 cAMP responsive element binding | I | 1 |
| protein 3 | | |
| CUL1 Cullin 1 | I | 1 |
| CXorf39 chromosome X open reading frame | I | 1 |
| 39 | | |
| DHRS12 dehydrogenase/reductase (SDR | I | 1 |
| family) member 12 | | |
| DIP2C DIP2 disco-interacting protein 2 | I | 1 |
| homolog C (Drosophila) | | |
| DNAH1 dynein, axonemal, heavy chain 1 | I | 1 |
| DNAI1 dynein, axonemal, intermediate chain 1 | I | 1 |
| Dock5 dedicator of cytokinesis 5 | I | 1 |
| ERN1 endoplasmic reticulum to nucleus | I | 1 |
| signaling 1 | | |
| FLJ12993 hypothetical LOC441027 | I | 1 |
| FLYWCH2 FLYWCH family member 2 | I | 1 |
| FSTL3 follistatin-like 3 (secreted glycoprotein) | I | 1 |
| GCS1 glucosidase I | I | 1 |
| GPNMB glycoprotein (transmembrane) nmb | I | 1 |
| GTF3C1 general transcription factor IIIC, | I | 1 |
| polypeptide 1, alpha 220 kDa | | |
| H2AFX H2A histone family, member X | I | 1 |
| Hnt Neurotrimin | I | 1 |
| IGHG1 Immunoglobulin heavy constant | I | 1 |
| gamma 1 (G1m marker) | | |
| ITIH4 inter-alpha (globulin) inhibitor H4 | I | 1 |
| (plasma Kallikrein-sensitive glycoprotein) | | |
| KHK ketohexokinase (fructokinase) /// | I | 1 |
| ketohexokinase (fructokinase) | | |
| KIAA1109 KIAA1109 | I | 1 |
| KRTAP8-1 keratin associated protein 8-1 | I | 1 |
| L3MBTL4 I(3)mbt-like 4 (Drosophila) | I | 1 |
| LMLN leishmanolysin-like (metallopeptidase | I | 1 |
| M8 family) | | |
| LOC284930 Hypothetical protein LOC284930 | I | 1 |
| LOC286144 hypothetical protein LOC286144 | I | 1 |
| LOC348174 secretory protein LOC348174 | I | 1 |
| LOC401074 hypothetical LOC401074 | I | 1 |
| LOC492311 similar to bovine IgA regulatory | I | 1 |
| protein | | |
| LOC645158 hypothetical protein LOC645158 | I | 1 |
| LOC645513 Similar to septin 7 | I | 1 |
| LOC728344 similar to Thioredoxin-like protein | I | 1 |
| 2 (PKC-interacting cousin of thioredoxin) | | |
| (PKC-theta-interacting protein) (PKCq- | | |
| interacting protein) | | |
| LOC731139 hypothetical protein LOC731139 | I | 1 |
| LRP3 low density lipoprotein receptor-related | I | 1 |
| protein 3 | | |
| MCTP2 multiple C2 domains, transmembrane 2 | I | 1 |
| MED1 mediator complex subunit 1 | I | 1 |
| MGC40499 PRotein Associated with Tlr4 | I | 1 |
| MMRN2 multimerin 2 | I | 1 |
| NLGN3 neuroligin 3 | I | 1 |
| NLRP3 NLR family, pyrin domain containing 3 | I | 1 |
| NPAL2 NIPA-like domain containing 2 | I | 1 |
| NUDT10 nudix (nucleoside diphosphate | I | 1 |
| linked moiety X)-type motif 10 | | |
| OR5T2 /// RPAIN RPA interacting protein /// | I | 1 |
| olfactory receptor, family 5, subfamily T, | | |
| member 2 | | |
| PARP15 poly (ADP-ribose) polymerase | I | 1 |
| family, member 15 | | |
| PDE3B Phosphodiesterase 3B, cGMP- | I | 1 |
| inhibited | | |
| PLEKHK1 pleckstrin homology domain | I | 1 |
| containing, family K member 1 | | |
| POU2F2 POU class 2 homeobox 2 | I | 1 |
| PSMB1 Proteasome (prosome, macropain) | I | 1 |
| subunit, beta type, 1 | | |
| QRSL1 | I | 1 |
| RAB23 RAB23, member RAS oncogene | I | 1 |
| family | | |
| RCN3 reticulocalbin 3, EF-hand calcium | I | 1 |
| binding domain | | |
| RHBDD3 rhomboid domain containing 3 | I | 1 |
| RNF165 ring finger protein 165 | I | 1 |
| RP2 retinitis pigmentosa 2 (X-linked | I | 1 |
| recessive) | | |
| RUFY2 RUN and FYVE domain containing 2 | I | 1 |
| SLC7A9 solute carrier family 7 (cationic | I | 1 |
| amino acid transporter, y+ system), member 9 | | |
| SMPD2 sphingomyelin phosphodiesterase 2, | I | 1 |
| neutral membrane (neutral sphingomyelinase) | | |
| SNHG10 Small nucleolar RNA host gene | I | 1 |
| (non-protein coding) 10 | | |
| SPATA2 spermatogenesis associated 2 | I | 1 |
| SPTY2D1 SPT2, Suppressor of Ty, domain | I | 1 |
| containing 1 (S. cerevisiae) | | |
| SUZ12P Suppressor of zeste 12 homolog | I | 1 |
| pseudogene | | |
| TAOK2 TAO kinase 2 | I | 1 |
| TCF7L1 transcription factor 7-like 1 (T-cell | I | 1 |
| specific, HMG-box) | | |
| TERF2 telomeric repeat binding factor 2 | I | 1 |
| THNSL1 threonine synthase-like 1 (S. cerevisiae) | I | 1 |
| Tpr translocated promoter region (to activated | I | 1 |
| MET oncogene) | | |
| TTC5 tetratricopeptide repeat domain 5 | I | 1 |
| USP6 ubiquitin specific protease 6 (Tre-2 | I | 1 |
| oncogene) | | |
| VILL villin-like | I | 1 |
| XDH xanthine dehydrogenase | I | 1 |
| YLPM1 YLP motif containing 1 | I | 1 |
| ZNF25 zinc finger protein 25 | I | 1 |
| ZNF546 zinc finger protein 546 | I | 1 |
| ZNF579 zinc finger protein 579 | I | 1 |
| ZNF597 zinc finger protein 597 | I | 1 |
| ZNF709 zinc finger protein 709 | I | 1 |
| ZNF746 zinc finger protein 746 | I | 1 |
| ZNHIT2 zinc finger, HIT type 2 | I | 1 |

TABLE 5B
Complete list of candidate biomarker genes for delusions
(n = 420) identified using A/P analysis and CFG scoring

| | Human Blood | CFG |
|---|---|---|
| Gene Symbol/Name | Delusions | Score |
| Drd2 dopamine receptor 2 | D | 6 |
| Egr1 early growth response 1 | I (HT) | 6 |
| APOE Apolipoprotein E | D | 5 |
| NRG1 neuregulin 1 | I | 5 |
| NAB1 NGFI-A binding protein 1 (EGR1 | D | 5 |
| binding protein 1) | | |
| IDH1 Isocitrate dehydrogenase 1 (NADP+), | D | 5 |
| soluble | | |
| Scamp1 secretory carrier membrane protein 1 | D | 5 |
| DCTN1 dynactin 1 (p150, glued homolog, | I (HT) | 5 |
| Drosophila) | | |
| TOB2 transducer of ERBB2, 2 | I (HT) | 5 |
| NCOA2 Nuclear receptor coactivator 2 | D | 4 |
| ALDH1L1 aldehyde dehydrogenase 1 family, | D | 4 |
| member L1 | | |
| Gpm6b Glycoprotein M6B | D | 4 |
| NMT1 N-myristoyltransferase 1 | I | 4 |
| Pctk1 PCTAIRE-motif protein kinase 1 | I | 4 |
| Pllp plasma membrane proteolipid | I | 4 |
| (plasmolipin) | | |
| Pvalb parvalbumin | I | 4 |
| Stxbp6 syntaxin binding protein 6 (amisyn) | D | 3 |
| PTPRM protein tyrosine phosphatase, | I | 3 |
| receptor type, M | | |
| ADAM9 ADAM metallopeptidase domain 9 | D | 3 |
| (meltrin gamma) | | |
| Fn1 fibronectin 1 | D | 3 |
| Add2 adducin 2 (beta) | D | 3 |
| Clasp1 CLIP associating protein 1 | D | 3 |
| Herc3 hect domain and RLD 3 | D | 3 |
| Mat2b methionine adenosyltransferase II, | D | 3 |
| beta | | |
| Mpp5 membrane protein, palmitoylated 5 | D | 3 |
| (MAGUK p55 subfamily member 5) | | |
| Prickle1 prickle like 1 (Drosophila) | D | 3 |
| Sh3bp4 SH3-domain binding protein 4 | D | 3 |
| Tmem106b transmembrane protein 106B | D | 3 |
| YAF2 YY1 associated factor 2 | D | 3 |
| Gpm6a glycoprotein m6a | D (HT) | 3 |
| Mgea5 Meningioma expressed antigen 5 | D (HT) | 3 |
| (hyaluronidase) | | |
| MRPL39 Mitochondrial ribosomal protein L39 | D (HT) | 3 |
| Adamts5 ADAM metallopeptidase with | I | 3 |
| thrombospondin type 1 motif, 5 | | |
| (aggrecanase-2) | | |
| Adamts5 ADAM metallopeptidase with | I | 3 |
| thrombospondin type 1 motif, 5 | | |
| (aggrecanase-2) | | |
| Cd84 CD84 molecule | I | 3 |
| Dnm2 dynamin 2 | I | 3 |
| Hbp1 HMG-box transcription factor 1 | I | 3 |
| KIF5C kinesin family member 5C | I | 3 |
| LTBP3 latent transforming growth factor beta | I | 3 |
| binding protein 3 | | |
| Ppap2a phosphatidic acid phosphatase 2a | I | 3 |
| Sparc secreted protein, acidic, cysteine-rich | I | 3 |
| (osteonectin) | | |
| VANGL1 vang-like 1 (van gogh, Drosophila) | I | 3 |
| BIN3 bridging integrator 3 | I (HT) | 3 |
| Gas2l1 growth arrest-specific 2 like 1 | I (HT) | 3 |
| HFE hemochromatosis | I (HT) | 3 |
| MFRP membrane frizzled-related protein | I (HT) | 3 |
| RCC1 /// SNHG3-RCC1 regulator of | I (HT) | 3 |
| chromosome condensation 1 | | |
| TP53I11 tumor protein p53 inducible protein | I (HT) | 3 |
| 11 | | |
| TPP1 tripeptidyl peptidase I | I (HT) | 3 |
| PTK2 PTK2 protein tyrosine kinase 2 | D | 2 |
| HIPK3 homeodomain interacting protein | I | 2 |
| kinase 3 | | |
| Actr3b ARP3 actin-related protein 3 homolog | D | 2 |
| B (yeast) | | |
| Angel2 angel homolog 2 (Drosophila) | D | 2 |
| Arid1b AT rich interactive domain 1B (SWI1- | D | 2 |
| like) | | |
| Atxn1 Ataxin 1 | D | 2 |
| B3galt2 UDP-Gal:betaGlcNAc beta 1,3- | D | 2 |
| galactosyltransferase, polypeptide 2 | | |
| Bckdhb branched chain keto acid | D | 2 |
| dehydrogenase E1, beta polypeptide (maple | | |
| syrup urine disease | | |
| Bhlhb3 basic helix-loop-helix domain | D | 2 |
| containing, class B, 3 | | |
| Bsdc1 BSD domain containing 1 | D | 2 |
| C10orf4 chromosome 10 open reading frame 4 | D | 2 |
| C10orf4 chromosome 10 open reading frame 4 | D | 2 |
| C1orf96 chromosome 1 open reading frame | D | 2 |
| 96 | | |
| Calml4 calmodulin-like 4 | D | 2 |
| Camsap1l1 calmodulin regulated spectrin- | D | 2 |
| associated protein 1-like 1 | | |
| Fam70a family with sequence similarity 70, | D | 2 |
| member A | | |
| Hbe1 hemoglobin, epsilon 1 | D | 2 |
| Hsf2 heat shock transcription factor 2 | D | 2 |
| INTS7 integrator complex subunit 8 | D | 2 |
| Loc285831 hypothetical protein LOC285831 | D | 2 |
| Lrch1 leucine-rich repeats and calponin | D | 2 |
| homology (CH) domain containing 1 | | |
| Map4k5 mitogen-activated protein kinase | D | 2 |
| kinase kinase kinase 5 /// mitogen-activated | | |
| protein kinase kinase kinase kinase 5 | | |
| March7 membrane-associated ring finger | D | 2 |
| (C3HC4) 7 | | |
| MYEF2 myelin expression factor 2 | D | 2 |
| Phf3 PHD finger protein 3 | D | 2 |
| Slc13a4 solute carrier family 13 | D | 2 |
| (sodium/sulfate symporters), member 4 | | |
| Tcf4 transcription factor 4 | D | 2 |
| Tmem64 transmembrane protein 64 | D | 2 |
| Tsc22d3 TSC22 domain family, member 3 | D | 2 |
| Twf1 twinfilin, actin-binding protein, homolog | D | 2 |
| 1 (Drosophila) | | |
| Zcchc12 zinc finger, CCHC domain | D | 2 |
| containing 12 | | |
| BCORL1 BCL6 co-repressor-like 1 | D (HT) | 2 |
| FAM137A family with sequence similarity 137, | D (HT) | 2 |
| member A | | |
| IQCH IQ motif containing H | D (HT) | 2 |
| MGC16075 hypothetical protein MGC16075 | D (HT) | 2 |
| NAV2 neuron navigator 2 | D (HT) | 2 |
| PLS3 plastin 3 (T isoform) | D (HT) | 2 |
| Adarb1 adenosine deaminase, RNA-specific, | I | 2 |
| B1 | | |
| Apcdd1 adenomatosis polyposis coli down- | I | 2 |
| regulated 1 | | |
| Atp2c1 ATPase, Ca++-sequestering | I | 2 |
| Camkv CaM kinase-like vesicle-associated | I | 2 |
| Dock9 Dedicator of cytokinesis 9 | I | 2 |
| Egln3 EGL nine homolog 3 (C. elegans) | I | 2 |
| Fosl2 FOS-like antigen 2 | I | 2 |
| Gnrhr gonadotropin-releasing hormone | I | 2 |
| receptor | | |
| Gp9 glycoprotein 9 (platelet) | I | 2 |
| Loc253039 hypothetical protein LOC253039 | I | 2 |
| MGC2752 Hypothetical protein MGC2752 | I | 2 |
| MGST1 microsomal glutathione S-transferase 1 | I | 2 |
| Pacs2 phosphofurin acidic cluster sorting | I | 2 |
| protein 2 | | |
| Plxnd1 Plexin D1 | I | 2 |
| PSD4 pleckstrin and Sec7 domain containing 4 | I | 2 |
| RANGAP1 Ran GTPase activating protein 1 | I | 2 |
| RANGAP1 Ran GTPase activating protein 1 | I | 2 |
| Sfxn5 sideroflexin 5 | I | 2 |
| SNX21 sorting nexin family member 21 | I | 2 |
| Stk32c serine/threonine kinase 32C | I | 2 |
| Tmem158 transmembrane protein 158 | I | 2 |
| Tmtc2 transmembrane and tetratricopeptide | I | 2 |
| repeat containing 2 | | |
| USP53 ubiquitin specific peptidase 53 | I | 2 |
| ZFP36L1 zinc finger protein 36, C3H type-like 1 | I | 2 |
| C19orf55 chromosome 19 open reading | I (HT) | 2 |
| frame 55 | | |
| GPR84 G protein-coupled receptor 84 | I (HT) | 2 |
| KIR2DS2 /// KIR2DS3 /// KIR2DS4 killer cell | I (HT) | 2 |
| immunoglobulin-like receptor, two domains, | | |
| short cytoplasmic tail, 2 /// killer cell | | |
| immunoglobulin-like receptor, two domains, | | |
| short cytoplasmic tail, 3 /// killer cell | | |
| immunoglobulin-like receptor, two domains, | | |
| short cytoplasmic tail, 4 | | |
| KLK2 kallikrein-related peptidase 2 | I (HT) | 2 |
| LOC90835 hypothetical protein LOC90835 | I (HT) | 2 |
| THTPA Thiamine triphosphatase | I (HT) | 2 |
| ABCA13 ATP-binding cassette, sub-family A | D | 1 |
| (ABC1), member 13 | | |
| ABCB4 ATP-binding cassette, sub-family B | D | 1 |
| (MDR/TAP), member 4 | | |
| ACRV1 acrosomal vesicle protein 1 | D | 1 |
| AFAP1L2 actin filament associated protein 1- | D | 1 |
| like 2 | | |
| ALS2CR4 amyotrophic lateral sclerosis 2 | D | 1 |
| (juvenile) chromosome region, candidate 4 | | |
| ARL17 ADP-ribosylation factor-like 17 | D | 1 |
| ARL4C ADP-ribosylation factor-like 4C | D | 1 |
| ASCC3 activating signal cointegrator 1 | D | 1 |
| complex subunit 3 | | |
| ATP9B ATPase, Class II, type 9B | D | 1 |
| BHLHB9 basic helix-loop-helix domain | D | 1 |
| containing, class B, 9 | | |
| BIRC7 baculoviral IAP repeat-containing 7 | D | 1 |
| (livin) | | |
| C17orf56 chromosome 17 open reading | D | 1 |
| frame 56 | | |
| C1orf114 chromosome 1 open reading frame | D | 1 |
| 114 | | |
| C1orf128 chromosome 1 open reading frame | D | 1 |
| 128 | | |
| C20orf42 chromosome 20 open reading | D | 1 |
| frame 42 | | |
| C2orf59 chromosome 2 open reading frame | D | 1 |
| 59 | | |
| C4orf18 chromosome 4 open reading frame | D | 1 |
| 18 | | |
| C4orf39 chromosome 4 open reading frame | D | 1 |
| 39 | | |
| C6orf114 chromosome 6 open reading frame | D | 1 |
| 114 | | |
| C6orf162 chromosome 6 open reading frame | D | 1 |
| 162 | | |
| C9orf117 chromosome 9 open reading frame | D | 1 |
| 117 | | |
| C9orf122 chromosome 9 open reading frame | D | 1 |
| 122 | | |
| CCDC117 coiled-coil domain containing 117 | D | 1 |
| CDK2 cyclin-dependent kinase 2 | D | 1 |
| CEP68 centrosomal protein 68 kDa | D | 1 |
| CLEC12A C-type lectin domain family 12, | D | 1 |
| member A | | |
| CLECL1 C-type lectin-like 1 | D | 1 |
| COL13A1 collagen, type XIII, alpha 1 | D | 1 |
| COPS8 COP9 constitutive photomorphogenic | D | 1 |
| homolog subunit 8 (Arabidopsis) | | |
| CYP2C9 cytochrome P450, family 2, | D | 1 |
| subfamily C, polypeptide 9 | | |
| DDR2 Discoidin domain receptor family, | D | 1 |
| member 2 | | |
| DDX3X /// DDX3Y DEAD (Asp-Glu-Ala-Asp) | D | 1 |
| box polypeptide 3, X-linked /// DEAD (Asp- | | |
| Glu-Ala-Asp) box polypeptide 3, Y-linked | | |
| DENND4C DENN/MADD domain containing | D | 1 |
| 4C | | |
| DNAJC11 DnaJ (Hsp40) homolog, subfamily | D | 1 |
| C, member 11 | | |
| DND1 dead end homolog 1 (zebrafish) | D | 1 |
| E2f5 E2F transcription factor 5, p130-binding | D | 1 |
| ENOX1 ecto-NOX disulfide-thiol exchanger 1 | D | 1 |
| EPM2A epilepsy, progressive myoclonus type | D | 1 |
| 2A, Lafora disease (laforin) | | |
| EPR1 Effector cell peptidase receptor 1 | D | 1 |
| ERGIC2 ERGIC and golgi 2 | D | 1 |
| ERICH1 Glutamate-rich 1 | D | 1 |
| Fa2h fatty acid 2-hydroxylase | D | 1 |
| FAM122C Family with sequence similarity | D | 1 |
| 122C | | |
| FAM82B Family with sequence similarity 82, | D | 1 |
| member B | | |
| FAM84A Family with sequence similarity 84, | D | 1 |
| member A | | |
| FANCI Fanconi anemia, complementation | D | 1 |
| group I | | |
| Fgf13 fibroblast growth factor 13 | D | 1 |
| FGF18 Fibroblast growth factor 18 | D | 1 |
| FGF7 /// KGFLP1 /// KGFLP2 fibroblast | D | 1 |
| growth factor 7 (keratinocyte growth factor) /// | | |
| keratinocyte growth factor-like protein 1 /// | | |
| keratinocyte growth factor-like protein 2 | | |
| FKBP7 FK506 binding protein 7 | D | 1 |
| FLJ13773 FLJ13773 | D | 1 |
| FLJ14082 hypothetical protein FLJ14082 | D | 1 |
| FLJ20323 hypothetical protein FLJ20323 | D | 1 |
| FOXD2 forkhead box D2 | D | 1 |
| FREM1 FRAS1 related extracellular matrix 1 | D | 1 |
| Fsd1l Fibronectin type III and SPRY domain | D | 1 |
| containing 1-like | | |
| FZD6 frizzled homolog 6 (Drosophila) | D | 1 |
| GABPA GA binding protein transcription | D | 1 |
| factor, alpha subunit 60 kDa | | |
| GLB1L3 galactosidase, beta 1 like 3 | D | 1 |
| GPR126 G protein-coupled receptor 126 | D | 1 |
| GPR23 G protein-coupled receptor 23 | D | 1 |
| GTPBP8 GTP-binding protein 8 (putative) | D | 1 |
| hCG_38480 potassium channel | D | 1 |
| tetramerisation domain containing 1 | | |
| HCRP1 hepatocellular carcinoma-related | D | 1 |
| HCRP1 | | |
| HERC4 hect domain and RLD 4 | D | 1 |
| HLA-DOA major histocompatibility complex, | D | 1 |
| class II, DO alpha | | |
| HUS1B HUS1 checkpoint homolog b (S. pombe) | D | 1 |
| IL5RA interleukin 5 receptor, alpha | D | 1 |
| ITFG1 integrin alpha 2b | D | 1 |
| JAKMIP2 jumonji, AT rich interactive domain | D | 1 |
| 1B | | |
| KLHL29 kelch-like 29 (Drosophila) | D | 1 |
| KRTAP4-9 keratin associated protein 4-9 | D | 1 |
| LGALS14 lectin, galactoside-binding, soluble, | D | 1 |
| 14 | | |
| LOC144874 | D | 1 |
| Loc158863 hypothetical protein LOC158863 | D | 1 |
| LOC221442 hypothetical LOC221442 | D | 1 |
| LOC255512 hypothetical protein LOC255512 | D | 1 |
| LOC256021 hypothetical protein LOC256021 | D | 1 |
| LOC388907 /// LOC642146 /// LOC647436 /// | D | 1 |
| RPL5 /// SNORA66 ribosomal protein L5 /// | | |
| small nucleolar RNA, H/ACA box 66 /// similar | | |
| to ribosomal protein L5 | | |
| LOC400581 GRB2-related adaptor protein- | D | 1 |
| like | | |
| LOC401913 hypothetical LOC401913 | D | 1 |
| LOC643837 hypothetical protein LOC643837 | D | 1 |
| LOC645431 hypothetical protein LOC645431 | D | 1 |
| LOC646677 /// LOC650674 similar to | D | 1 |
| aconitase 2, mitochondrial | | |
| LOC730961 hypothetical protein LOC730961 | D | 1 |
| LOC92497 hypothetical protein LOC92497 | D | 1 |
| LYZL6 lysozyme-like 6 | D | 1 |
| MAGIX MAGI family member, X-linked | D | 1 |
| MARVELD2 MARVEL domain containing 2 | D | 1 |
| MDM1 Mdm4, transformed 3T3 cell double | D | 1 |
| minute 1, p53 binding protein (mouse) | | |
| MGC22265 hypothetical protein MGC22265 | D | 1 |
| MREG melanoregulin | D | 1 |
| MUC3A mucin 3A, cell surface associated | D | 1 |
| NDUFB7 NADH dehydrogenase (ubiquinone) | D | 1 |
| 1 beta subcomplex, 7, 18 kDa | | |
| NHEDC1 | D | 1 |
| OSGEP O-sialoglycoprotein endopeptidase | D | 1 |
| OVOL1 ovo-like 1(Drosophila) | D | 1 |
| PCOLCE2 procollagen C-endopeptidase | D | 1 |
| enhancer 2 | | |
| PFDN4 prefoldin subunit 4 | D | 1 |
| PGAP1 GPI deacylase | D | 1 |
| PID1 phosphotyrosine interaction domain | D | 1 |
| containing 1 | | |
| PIK3C2A phosphoinositide-3-kinase, class 2, | D | 1 |
| alpha polypeptide | | |
| PMS1 PMS1 postmeiotic segregation | D | 1 |
| increased 1 (S. cerevisiae) | | |
| PNO1 partner of NOB1 homolog (S. cerevisiae) | D | 1 |
| PPP2R1B protein phosphatase 2 (formerly | D | 1 |
| 2A), regulatory subunit A, beta isoform | | |
| PROM2 prominin 2 | D | 1 |
| PSG6 pregnancy specific beta-1-glycoprotein 6 | D | 1 |
| RBM11 RNA binding motif protein 11 | D | 1 |
| REPS1 RALBP1 associated Eps domain | D | 1 |
| containing 1 | | |
| RP4-662A9.2 hypothetical protein MGC34034 | D | 1 |
| RPESP RPE-spondin | D | 1 |
| RUFY1 RUN and FYVE domain containing 1 | D | 1 |
| SEC16B SEC16 homolog B (S. cerevisiae) | D | 1 |
| SENP7 SUMO1/sentrin specific peptidase 7 | D | 1 |
| SLAIN2 SLAIN motif family, member 2 | D | 1 |
| SLC2A5 solute carrier family 2 (facilitated | D | 1 |
| glucose/fructose transporter), member 5 | | |
| SLC35B3 solute carrier family 35, member B3 | D | 1 |
| SLC37A3 solute carrier family 37 (glycerol-3- | D | 1 |
| phosphate transporter), member 3 | | |
| SMC3 Structural maintenance of | D | 1 |
| chromosomes 3 | | |
| SPAG11A /// SPAG11B sperm associated | D | 1 |
| antigen 11B /// sperm associated antigen 11A | | |
| SPAG8 sperm associated antigen 8 | D | 1 |
| SPINK2 serine peptidase inhibitor, Kazal type | D | 1 |
| 2 (acrosin-trypsin inhibitor) | | |
| STXBP4 syntaxin binding protein 4 | D | 1 |
| STYK1 serine/threonine/tyrosine kinase 1 | D | 1 |
| SYCP3 synaptonemal complex protein 3 | D | 1 |
| TFG TRK-fused gene | D | 1 |
| TMEM174 transmembrane protein 174 | D | 1 |
| TMEM38B transmembrane protein 38B | D | 1 |
| TNRC6C trinucleotide repeat containing 6C | D | 1 |
| TRIM48 tripartite motif-containing 48 | D | 1 |
| UBE4B Ubiquitination factor E4B (UFD2 | D | 1 |
| homolog, yeast) | | |
| UPP2 uridine phosphorylase 2 | D | 1 |
| VAPA VAMP (vesicle-associated membrane | D | 1 |
| protein)-associated protein A, 33 kDa | | |
| WBSCR23 Williams-Beuren syndrome | D | 1 |
| chromosome region 23 | | |
| WDR19 WD repeat domain 19 | D | 1 |
| ZFX zinc finger protein, X-linked | D | 1 |
| ZKSCAN3 zinc finger with KRAB and SCAN | D | 1 |
| domains 3 | | |
| ZKSCAN3 zinc finger with KRAB and SCAN | D | 1 |
| domains 3 | | |
| ZNF146 zinc finger protein 146 | D | 1 |
| ZNF345 zinc finger protein 345 | D | 1 |
| ZNF441 zinc finger protein 441 | D | 1 |
| ZNF479 zinc finger protein 479 | D | 1 |
| Znf614 zinc finger protein 614 | D | 1 |
| ZNF675 zinc finger protein 675 | D | 1 |
| ZNF683 zinc finger protein 683 | D | 1 |
| ZNF789 zinc finger protein 789 | D | 1 |
| ZRANB2 zinc finger, RAN-binding domain | D | 1 |
| containing 2 | | |
| ABCC4 ATP-binding cassette, sub-family C | I | 1 |
| (CFTR/MRP), member 4 | | |
| Akap8l A kinase (PRKA) anchor protein 8-like | I | 1 |
| ARHGAP29 Rho GTPase activating protein | I | 1 |
| 29 | | |
| ATPAF2 ATP synthase mitochondrial F1 | I | 1 |
| complex assembly factor 2 | | |
| BACE1 beta-site APP-cleaving enzyme 1 | I | 1 |
| BACE1 beta-site APP-cleaving enzyme 1 | I | 1 |
| Bet3l BET3 like (S. cerevisiae | I | 1 |
| C10orf47 chromosome 10 open reading | I | 1 |
| frame 47 | | |
| C19orf48 chromosome 19 open reading | I | 1 |
| frame 48 | | |
| C7orf16 chromosome 7 open reading frame | I | 1 |
| 16 | | |
| C7orf34 chromosome 7 open reading frame | I | 1 |
| 34 | | |
| C8orf30A chromosome 8 open reading frame | I | 1 |
| 30A | | |
| CBL Cas-Br-M (murine) ecotropic retroviral | I | 1 |
| transforming sequence | | |
| CCNT1 cyclin T1 | I | 1 |
| CDC42EP2 CDC42 effector protein (Rho | I | 1 |
| GTPase binding) 2 | | |
| CDH23 cadherin-like 23 | I | 1 |
| CDK10 cyclin-dependent kinase (CDC2-like) | I | 1 |
| 10 | | |
| CHAF1A chromatin assembly factor 1, | I | 1 |
| subunit A (p150) | | |
| CLCC1 Chloride channel CLIC-like 1 | I | 1 |
| CLDN5 claudin 5 (transmembrane protein | I | 1 |
| deleted in velocardiofacial syndrome) | | |
| CLEC11A C-type lectin domain family 11, | I | 1 |
| member A | | |
| CLPTM1 cleft lip and palate associated | I | 1 |
| transmembrane protein 1 | | |
| Cltc clathrin, heavy polypeptide (Hc) | I | 1 |
| COPZ2 coatomer protein complex, subunit | I | 1 |
| zeta 2 | | |
| CPNE9 copine family member IX | I | 1 |
| CRISP2 cysteine-rich secretory protein 2 | I | 1 |
| CTNS cystinosis, nephropathic | I | 1 |
| CUL1 Cullin 1 | I | 1 |
| CUL7 cullin 7 | I | 1 |
| DAB2 disabled homolog 2, mitogen- | I | 1 |
| responsive phosphoprotein (Drosophila) | | |
| DHRS12 dehydrogenase/reductase (SDR | I | 1 |
| family) member 12 | | |
| DNAH1 dynein, axonemal, heavy chain 1 | I | 1 |
| DNAI1 dynein, axonemal, intermediate chain 1 | I | 1 |
| DNHD1 dynein heavy chain domain 1 | I | 1 |
| DNPEP aspartyl aminopeptidase | I | 1 |
| DTX2 deltex homolog 2 (Drosophila) | I | 1 |
| DUSP13 dual specificity phosphatase 13 | I | 1 |
| ERN1 endoplasmic reticulum to nucleus | I | 1 |
| signaling 1 | | |
| FAM129B family with sequence similarity 129, | I | 1 |
| member B | | |
| FLJ10241 Hypothetical protein FLJ10241 | I | 1 |
| FLJ12993 hypothetical LOC441027 | I | 1 |
| FLJ35348 FLJ35348 | I | 1 |
| FLT3 fms-related tyrosine kinase 3 | I | 1 |
| FSTL3 follistatin-like 3 (secreted glycoprotein) | I | 1 |
| GALE UDP-galactose-4-epimerase | I | 1 |
| GCS1 glucosidase I | I | 1 |
| GFI1B growth factor independent 1B | I | 1 |
| (potential regulator of CDKN1A, translocated | | |
| in CML) | | |
| GLT25D1 glycosyltransferase 25 domain | I | 1 |
| containing 1 | | |
| GPR157 G protein-coupled receptor 157 | I | 1 |
| GRAMD1B GRAM domain containing 1B | I | 1 |
| GTF3C1 general transcription factor IIIC, | I | 1 |
| polypeptide 1, alpha 220 kDa | | |
| H19 H19, imprinted maternally expressed | I | 1 |
| untranslated mRNA | | |
| HBBP1 hemoglobin, beta pseudogene 1 | I | 1 |
| HMG20B high-mobility group 20B | I | 1 |
| HOXA11S homeo box A11, antisense | I | 1 |
| IER5L immediate early response 5-like | I | 1 |
| JMJD3 jumonji domain containing 3 | I | 1 |
| KCNE1 potassium voltage-gated channel, | I | 1 |
| Isk-related family, member 1 | | |
| KCTD15 potassium channel tetramerisation | I | 1 |
| domain containing 15 | | |
| KHK ketohexokinase (fructokinase) /// | I | 1 |
| ketohexokinase (fructokinase) | | |
| KIAA0319L KIAA0319-like | I | 1 |
| KIAA1602 KIAA1602 | I | 1 |
| KIAA1856 KIAA1856 protein | I | 1 |
| KIF13A kinesin family member 13A | I | 1 |
| KIF9 kinesin family member 9 | I | 1 |
| KIR2DL4 killer cell immunoglobulin-like | I | 1 |
| receptor, two domains, long cytoplasmic tail, 4 | | |
| KIR2DL5A killer cell immunoglobulin-like | I | 1 |
| receptor, two domains, long cytoplasmic tail, | | |
| 5A | | |
| KLHL14 kelch-like 14 (Drosophila) | I | 1 |
| KRIT1 KRIT1, ankyrin repeat containing | I | 1 |
| LASS4 LAG1 homolog, ceramide synthase 4 | I | 1 |
| LOC147650 /// LOC729781 hypothetical | I | 1 |
| protein LOC147650 /// hypothetical protein | | |
| LOC729781 | | |
| LOC151657 hypothetical protein LOC151657 | I | 1 |
| LOC162073 hypothetical protein LOC162073 | I | 1 |
| LOC253842 /// NR6A1 nuclear receptor | I | 1 |
| subfamily 6, group A, member 1 /// | | |
| hypothetical protein LOC253842 | | |
| LOC254100 hypothetical protein LOC254100 | I | 1 |
| LOC283050 hypothetical protein LOC283050 | I | 1 |
| Loc283481 hypothetical protein LOC283481 | I | 1 |
| LOC283922 /// LOC650883 /// LOC651987 /// | I | 1 |
| PDPR pyruvate dehydrogenase phosphatase | | |
| regulatory subunit /// hypothetical protein | | |
| LOC283922 /// similar to pyruvate | | |
| dehydrogenase phosphatase regulatory | | |
| subunit | | |
| LOC348174 secretory protein LOC348174 | I | 1 |
| LOC389831 Hypothetical gene supported by | I | 1 |
| AL713796 | | |
| LOC400960 Hypothetical gene supported by | I | 1 |
| BC040598 | | |
| LOC442262 /// LOC732268 similar to | I | 1 |
| Glyceraldehyde-3-phosphate dehydrogenase | I | 1 |
| (GAPDH) | | |
| LOC645166 similar to lymphocyte-specific | I | 1 |
| protein 1 isoform 1 | | |
| LRP3 low density lipoprotein receptor-related | I | 1 |
| protein 3 | | |
| LRRC37A3 Leucine rich repeat containing 37, | I | 1 |
| member A3 | | |
| Lrrc4 leucine rich repeat containing 4 | I | 1 |
| LRRC8A leucine rich repeat containing 8 | I | 1 |
| family, member A | | |
| LTA lymphotoxin alpha (TNF superfamily, | I | 1 |
| member 1) | | |
| March9 membrane-associated ring finger | I | 1 |
| (C3HC4) 9 | | |
| MCM3AP | I | 1 |
| MCTP2 multiple C2 domains, transmembrane 2 | I | 1 |
| MED1 mediator complex subunit 1 | I | 1 |
| MGC40499 PRotein Associated with Tlr4 | I | 1 |
| MLCK MLCK protein | I | 1 |
| MSRB3 methionine sulfoxide reductase B3 | I | 1 |
| MXD1 MAX dimerization protein 1 | I | 1 |
| MYO7A myosin VIIA | I | 1 |
| MYST4 MYST histone acetyltransferase | I | 1 |
| (monocytic leukemia) 4 | | |
| NKAPL NFKB activating protein-like | I | 1 |
| NT5M 5′,3′-nucleotidase, mitochondrial | I | 1 |
| NUP188 nucleoporin 188 kDa | I | 1 |
| OAF OAF homolog (Drosophila) | I | 1 |
| OPA3 optic atrophy 3 (autosomal recessive, | I | 1 |
| with chorea and spastic paraplegia) | | |
| PCLKC protocadherin LKC | I | 1 |
| PDE3B Phosphodiesterase 3B, cGMP- | I | 1 |
| inhibited | | |
| PHACTR1 phosphatase and actin regulator 1 | I | 1 |
| PHEX phosphate regulating endopeptidase | I | 1 |
| homolog, X-linked (hypophosphatemia, | | |
| vitamin D resistant rickets) | | |
| PKP4 plakophilin 4 | I | 1 |
| PLD1 phospholipase D1, | I | 1 |
| phosphatidylcholine-specific | | |
| PLEKHG2 pleckstrin homology domain | I | 1 |
| containing, family G (with RhoGef domain) | | |
| member 2 | | |
| PML promyelocytic leukemia | I | 1 |
| PPM1H protein phosphatase 1H (PP2C | I | 1 |
| domain containing) | | |
| PPP1R10 protein phosphatase 1, regulatory | I | 1 |
| (inhibitor) subunit 10 | | |
| PPP2R3B protein phosphatase 2 (formerly | I | 1 |
| 2A), regulatory subunit B″, beta | | |
| PRRT2 proline-rich transmembrane protein 2 | I | 1 |
| PSMB1 Proteasome (prosome, macropain) | I | 1 |
| subunit, beta type, 1 | | |
| PTCD1 pentatricopeptide repeat domain 1 | I | 1 |
| RAB15 RAB15, member RAS onocogene | I | 1 |
| family | | |
| RAD54L2 RAD54-like 2 (S. cerevisiae) | I | 1 |
| RBL1 retinoblastoma-like 1 (p107) | I | 1 |
| RHAG Rh-associated glycoprotein | I | 1 |
| RNF122 ring finger protein 122 | I | 1 |
| SAMD4B Sterile alpha motif domain | I | 1 |
| containing 4B | | |
| SAPS2 SAPS domain family, member 2 | I | 1 |
| SCARB1 scavenger receptor class B, | I | 1 |
| member 1 | | |
| SCFD2 sec1 family domain containing 2 | I | 1 |
| SDCCAG8 serologically defined colon cancer | I | 1 |
| antigen 8 | | |
| Sergef Secretion regulating guanine | I | 1 |
| nucleotide exchange factor | | |
| SERPINB8 serpin peptidase inhibitor, clade B | I | 1 |
| (ovalbumin), member 8 | | |
| Sesn3 sestrin 3 | I | 1 |
| SIL1 SIL1 homolog, endoplasmic reticulum | I | 1 |
| chaperone (S. cerevisiae) | | |
| SLC22A1 solute carrier family 22 (organic | I | 1 |
| cation transporter), member 1 | | |
| SLC26A6 solute carrier family 26, member 6 | I | 1 |
| SLC39A3 solute carrier family 39 (zinc | I | 1 |
| transporter), member 3 | | |
| SLC39A3 solute carrier family 39 (zinc | I | 1 |
| transporter), member 3 | | |
| SMPD2 sphingomyelin phosphodiesterase 2, | I | 1 |
| neutral membrane (neutral sphingomyelinase) | | |
| SPHK2 sphingosine kinase 2 | I | 1 |
| SPSB1 splA/ryanodine receptor domain and | I | 1 |
| SOCS box containing 1 | | |
| SREBF1 sterol regulatory element binding | I | 1 |
| transcription factor 1 | | |
| STAT5B signal transducer and activator of | I | 1 |
| transcription 5B | | |
| STAT5B signal transducer and activator of | I | 1 |
| transcription 5B | | |
| TK2 thymidine kinase 2, mitochondrial | I | 1 |
| TMEM112B transmembrane protein 112B | I | 1 |
| TMEM8 transmembrane protein 8 (five | I | 1 |
| membrane-spanning domains) | | |
| TRIP13 thyroid hormone receptor interactor | I | 1 |
| 13 | | |
| TUT1 terminal uridylyl transferase 1, U6 | I | 1 |
| snRNA-specific | | |
| ULK2 Unc-51-like kinase 2 (C. elegans) | I | 1 |
| USP40 ubiquitin specific peptidase 40 | I | 1 |
| WDR24 WD repeat domain 24 | I | 1 |
| YLPM1 YLP motif containing 1 | I | 1 |
| ZAK sterile alpha motif and leucine zipper | I | 1 |
| containing kinase AZK | | |
| ZBTB38 zinc finger and BTB domain | I | 1 |
| containing 38 | | |
| ZNF107 Zinc finger protein 107 | I | 1 |
| ZNF25 zinc finger protein 25 | I | 1 |
| ZNF473 zinc finger protein 473 | I | 1 |
| Znf793 zinc finger protein 793 | I | 1 |
| ZNHIT2 zinc finger, HIT type 2 | I | 1 |

TABLE 6A
Additional candidate biomarker genes for hallucinations (n = 15) identified by
differential gene expression analysis (using p < 0.005).

| | | | | Fold Change | |
|---|---|---|---|---|---|
| | | | | (High | |
| | | | | hallucinations vs. | |
| Affymetrix | | | | No | |
| Probe set ID | Gene symbol | Gene name | Change | Hallucinations) | p-value |
| 203645_s_at | CD163 | CD163 molecule | I | 1.689014 | 0.002896 |
| 203940_s_at | VASH1 | vasohibin 1 | I | 1.598059 | 0.002503 |
| 219858_s_at | FLJ20160 | FLJ20160 protein | D | 0.817759 | 0.004154 |
| 244561_at | LOC169932 | Homo sapiens, | D | 0.803799 | 0.00124 |
| | | Similar to | | | |
| | | LOC169932, clone | | | |
| | | IMAGE: 4499203, | | | |
| | | mRNA | | | |
| 1560981_a_at | PPARA | peroxisome | D | 0.795834 | 0.001769 |
| | | proliferator-activated | | | |
| | | receptor alpha | | | |
| 1554696_s_at | TYMS | thymidylate | D | 0.792632 | 0.002091 |
| | | synthetase | | | |
| 232682_at | MREG | melanoregulin | D | 0.791137 | 0.003397 |
| 238585_at | GTDC1 | glycosyltransferase- | D | 0.783062 | 0.001631 |
| | | like domain | | | |
| | | containing 1 | | | |
| 1559745_at | FLJ34261 | CDNA FLJ34261 fis, | D | 0.768636 | 0.002779 |
| | | clone | | | |
| | | FEBRA2001772 | | | |
| 217551_at | LOC441453 | similar to olfactory | D | 0.75366 | 0.002353 |
| | | receptor, family 7, | | | |
| | | subfamily A, member | | | |
| | | 17 | | | |
| 205110_s_at | FGF13 | fibroblast growth | D | 0.739487 | 0.002837 |
| | | factor 13 | | | |
| 240671_at | CS0DM007YH16 | Full-length cDNA | D | 0.730351 | 0.003986 |
| | | clone | | | |
| | | CS0DM007YH16 of | | | |
| | | Fetal liver of Homo | | | |
| | | sapiens (human) | | | |
| 230120_s_at | PLGLB2 | plasminogen-like B2 | D | 0.635245 | 0.004878 |
| 213515_x_at | HBG1 | hemoglobin, gamma A | D | 0.601758 | 0.001206 |
| 230720_at | RNF182 | ring finger protein 182 | D | 0.220427 | 0.001301 |

I—increased in high hallucinations state (biomarker for high hallucinations);
D—decreased in high hallucinations state/increased in no hallucinations state (biomarker for no hallucinations).

TABLE 6B
Additional candidate biomarker genes for delusions (n = 132) identified by
differential gene expression analysis (using p < 0.005).

| | | | | Fold Change (High | |
|---|---|---|---|---|---|
| Affymetrix Probe | Gene | | | Delusions vs. No | |
| set ID | symbol | Gene name | Change | Delusions) | p-value |
| 201294_s_at | WSB1 | WD repeat and SOCS box- | I | 2.331482 | 0.001337 |
| | | containing 1 | | | |
| 201867_s_at | TBL1X | transducin (beta)-like 1X- | I | 2.264816 | 0.002198 |
| | | linked | | | |
| 209791_at | PADI2 | peptidyl arginine deiminase, | I | 2.122851 | 0.003888 |
| | | type II | | | |
| 205069_s_at | ARHGAP26 | Rho GTPase activating | I | 2.07213 | 0.004859 |
| | | protein 26 | | | |
| 201280_s_at | DAB2 | disabled homolog 2, mitogen- | I | 2.025662 | 0.000231 |
| | | responsive phosphoprotein | | | |
| | | (Drosophila) | | | |
| 231205_at | | | I | 1.945909 | 0.003222 |
| 207314_x_at | KIR3DL2 | killer cell immunoglobulin-like | I | 1.935486 | 0.000231 |
| | | receptor, three domains, long | | | |
| | | cytoplasmic tail, 2 | | | |
| 201224_s_at | SRRM1 | serine/arginine repetitive | I | 1.920132 | 0.004674 |
| | | matrix 1 | | | |
| 209811_at | CASP2 | caspase 2, apoptosis-related | I | 1.913978 | 0.004358 |
| | | cysteine peptidase (neural | | | |
| | | precursor cell expressed, | | | |
| | | developmentally down- | | | |
| | | regulated 2) | | | |
| 203645_s_at | CD163 | CD163 molecule | I | 1.886939 | 0.000383 |
| 1558397_at | | CDNA FLJ34100 fis, clone | I | 1.839954 | 0.000973 |
| | | FCBBF3007597 | | | |
| 211532_x_at | KIR2DS1 | killer cell immunoglobulin-like | I | 1.811797 | 0.002169 |
| | | receptor, two domains, short | | | |
| | | cytoplasmic tail, 1 | | | |
| 1559582_at | RHOQ | ras homolog gene family, | I | 1.799617 | 0.001452 |
| | | member Q | | | |
| 215087_at | C15orf39 | chromosome 15 open reading | I | 1.798779 | 0.000536 |
| | | frame 39 | | | |
| 225954_s_at | MIDN | midnolin | I | 1.798375 | 0.003021 |
| 215706_x_at | ZYX | zyxin | I | 1.788868 | 0.001005 |
| 210166_at | TLR5 | toll-like receptor 5 | I | 1.743849 | 0.000235 |
| 224992_s_at | CMIP | c-Maf-inducing protein | I | 1.724533 | 0.004533 |
| 239798_at | | Transcribed locus | I | 1.704032 | 0.001937 |
| 236094_at | TCF7L2 | Transcription factor 7-like 2 | I | 1.688487 | 0.002603 |
| | | (T-cell specific, HMG-box) | | | |
| 240775_at | | | I | 1.675145 | 0.002623 |
| 229373_at | | Transcribed locus | I | 1.66609 | 0.003067 |
| 241742_at | PRAM1 | PML-RARA regulated adaptor | I | 1.649353 | 0.002492 |
| | | molecule 1 | | | |
| 223466_x_at | COL4A3BP | collagen, type IV, alpha 3 | I | 1.647247 | 0.003274 |
| | | (Goodpasture antigen) binding | | | |
| | | protein | | | |
| 242106_at | | Transcribed locus | I | 1.643629 | 0.002849 |
| 201082_s_at | DCTN1 | dynactin 1 (p150, glued | I | 1.631231 | 0.004825 |
| | | homolog, Drosophila) | | | |
| 225234_at | CBL | Cas-Br-M (murine) ecotropic | I | 1.615207 | 0.002695 |
| | | retroviral transforming | | | |
| | | sequence | | | |
| 224818_at | SORT1 | sortilin 1 | I | 1.607849 | 0.001417 |
| 1565628_at | | Full length insert cDNA clone | I | 1.607681 | 0.002329 |
| | | ZD55G10 | | | |
| 200964_at | UBA1 | ubiquitin-like modifier | I | 1.606868 | 0.001323 |
| | | activating enzyme 1 | | | |
| 239701_at | | Transcribed locus | I | 1.6065 | 0.002877 |
| 200601_at | ACTN4 | actinin, alpha 4 | I | 1.60477 | 0.003227 |
| 201482_at | QSOX1 | quiescin Q6 sulfhydryl oxidase 1 | I | 1.583122 | 5.63E−05 |
| 210423_s_at | SLC11A1 | solute carrier family 11 | I | 1.581788 | 0.002722 |
| | | (proton-coupled divalent metal | | | |
| | | ion transporters), member 1 | | | |
| 200678_x_at | GRN | granulin | I | 1.581639 | 0.004041 |
| 201353_s_at | BAZ2A | bromodomain adjacent to zinc | I | 1.581437 | 0.001572 |
| | | finger domain, 2A | | | |
| 200752_s_at | CAPN1 | calpain 1, (mu/l) large subunit | I | 1.569854 | 0.00401 |
| 225280_x_at | ARSD | arylsulfatase D | I | 1.568151 | 0.000704 |
| 238996_x_at | ALDOA | aldolase A, fructose- | I | 1.563824 | 0.002864 |
| | | bisphosphate | | | |
| 204647_at | HOMER3 | homer homolog 3 (Drosophila) | I | 1.561796 | 0.002949 |
| 214755_at | UAP1L1 | UDP-N-acteylglucosamine | I | 1.558387 | 0.00374 |
| | | pyrophosphorylase 1-like 1 | | | |
| 201995_at | EXT1 | exostoses (multiple) 1 | I | 1.556286 | 0.000937 |
| 201050_at | PLD3 | phospholipase D family, | I | 1.555799 | 0.003185 |
| | | member 3 | | | |
| 205349_at | GNA15 | guanine nucleotide binding | I | 1.552415 | 0.000829 |
| | | protein (G protein), alpha 15 | | | |
| | | (Gq class) | | | |
| 215535_s_at | AGPAT1 | 1-acylglycerol-3-phosphate O- | I | 1.551735 | 0.002967 |
| | | acyltransferase 1 | | | |
| | | (lysophosphatidic acid | | | |
| | | acyltransferase, alpha) | | | |
| 41387_r_at | JMJD3 | jumonji domain containing 3, | I | 1.550806 | 0.004413 |
| | | histone lysine demethylase | | | |
| 206130_s_at | ASGR2 | asialoglycoprotein receptor 2 | I | 1.545796 | 0.004093 |
| 212334_at | GNS | glucosamine (N-acetyl)-6- | I | 1.543919 | 0.002239 |
| | | sulfatase (Sanfilippo disease | | | |
| | | IIID) | | | |
| 224374_s_at | EMILIN2 | elastin microfibril interfacer 2 | I | 1.540626 | 0.003506 |
| 200766_at | CTSD | cathepsin D | I | 1.540228 | 0.001848 |
| 224393_s_at | CECR6 | cat eye syndrome | I | 1.5395 | 0.00398 |
| | | chromosome region, | | | |
| | | candidate 6 | | | |
| 219382_at | SERTAD3 | SERTA domain containing 3 | I | 1.535854 | 0.002134 |
| 1552410_at | CLEC4F | C-type lectin domain family 4, | I | 1.532775 | 0.004005 |
| | | member F | | | |
| 214752_x_at | FLNA | filamin A, alpha (actin binding | I | 1.532563 | 0.001602 |
| | | protein 280) | | | |
| 212303_x_at | | | I | 1.522 | 0.002956 |
| 221006_s_at | SNX27 | sorting nexin family member | I | 1.516024 | 0.002948 |
| | | 27 | | | |
| 201536_at | DUSP3 | dual specificity phosphatase 3 | I | 1.511698 | 0.001187 |
| | | (vaccinia virus phosphatase | | | |
| | | VH1-related) | | | |
| 226139_at | | Full length insert cDNA clone | I | 1.509803 | 0.003546 |
| | | ZA04F06 | | | |
| 227853_at | | Transcribed locus, moderately | I | 1.506351 | 0.002921 |
| | | similar to NP_689672.2 | | | |
| | | hypothetical protein | | | |
| | | LOC146556 [Homo sapiens] | | | |
| 1554016_a_at | C16orf57 | chromosome 16 open reading | I | 1.491642 | 0.000986 |
| | | frame 57 | | | |
| 209367_at | STXBP2 | syntaxin binding protein 2 | I | 1.484629 | 0.004756 |
| 231963_at | | Homo sapiens, clone | I | 1.481555 | 0.001326 |
| | | IMAGE: 3869276, mRNA | | | |
| 237647_at | GHRL | Ghrelin/obestatin | I | 1.480238 | 0.004896 |
| | | preprohormone | | | |
| 204787_at | VSIG4 | V-set and immunoglobulin | I | 1.480102 | 0.003033 |
| | | domain containing 4 | | | |
| 64899_at | LPPR2 | lipid phosphate phosphatase- | I | 1.477132 | 0.003858 |
| | | related protein type 2 | | | |
| 200827_at | PLOD1 | procollagen-lysine 1,2- | I | 1.477079 | 0.001659 |
| | | oxoglutarate 5-dioxygenase 1 | | | |
| 216016_at | NLRP3 | NLR family, pyrin domain | I | 1.463122 | 0.002161 |
| | | containing 3 | | | |
| 221900_at | COL8A2 | collagen, type VIII, alpha 2 | I | 1.46288 | 0.004072 |
| 226728_at | SLC27A1 | solute carrier family 27 (fatty | I | 1.460467 | 0.000821 |
| | | acid transporter), member 1 | | | |
| 206380_s_at | CFP | complement factor properdin | I | 1.457147 | 0.003635 |
| 213113_s_at | SLC43A3 | solute carrier family 43, | I | 1.439744 | 0.002749 |
| | | member 3 | | | |
| 207233_s_at | MITF | microphthalmia-associated | I | 1.438848 | 0.002593 |
| | | transcription factor | | | |
| 205142_x_at | ABCD1 | ATP-binding cassette, sub- | I | 1.424328 | 0.00225 |
| | | family D (ALD), member 1 | | | |
| 1570151_at | | Homo sapiens, clone | I | 1.42162 | 0.002573 |
| | | IMAGE: 4340670, mRNA | | | |
| 210314_x_at | TNFSF13 | tumor necrosis factor (ligand) | I | 1.421376 | 0.003868 |
| | | superfamily, member 13 | | | |
| 211067_s_at | GAS7 | growth arrest-specific 7 | I | 1.417018 | 0.000716 |
| 202295_s_at | CTSH | cathepsin H | I | 1.415572 | 0.000592 |
| 213329_at | SRGAP2 | SLIT-ROBO Rho GTPase | I | 1.412918 | 0.00083 |
| | | activating protein 2 | | | |
| 203444_s_at | MTA2 | metastasis associated 1 | I | 1.41216 | 0.004281 |
| | | family, member 2 | | | |
| 208947_s_at | UPF1 | UPF1 regulator of nonsense | I | 1.406639 | 0.004512 |
| | | transcripts homolog (yeast) | | | |
| 213298_at | NFIC | nuclear factor I/C (CCAAT- | I | 1.404354 | 0.002383 |
| | | binding transcription factor) | | | |
| 1552553_a_at | NLRC4 | NLR family, CARD domain | I | 1.402 | 0.002664 |
| | | containing 4 | | | |
| 232724_at | MS4A6A | membrane-spanning 4- | I | 1.395938 | 0.000996 |
| | | domains, subfamily A, | | | |
| | | member 6A | | | |
| 218389_s_at | APH1A | anterior pharynx defective 1 | I | 1.394406 | 0.003529 |
| | | homolog A (C. elegans) | | | |
| 203330_s_at | STX5 | syntaxin 5 | I | 1.393468 | 0.000333 |
| 200871_s_at | PSAP | prosaposin (variant Gaucher | I | 1.392357 | 0.001125 |
| | | disease and variant | | | |
| | | metachromatic | | | |
| | | leukodystrophy) | | | |
| 202801_at | PRKACA | protein kinase, cAMP- | I | 1.38035 | 0.003856 |
| | | dependent, catalytic, alpha | | | |
| 225592_at | NRM | nurim (nuclear envelope | I | 1.380165 | 0.003149 |
| | | membrane protein) | | | |
| 217782_s_at | GPS1 | G protein pathway suppressor 1 | I | 1.367781 | 0.004758 |
| 221807_s_at | TRABD | TraB domain containing | I | 1.362005 | 0.003895 |
| 222469_s_at | TOLLIP | toll interacting protein | I | 1.360515 | 0.002874 |
| 206219_s_at | VAV1 | vav 1 guanine nucleotide | I | 1.359847 | 0.002254 |
| | | exchange factor | | | |
| 213448_at | | CDNA FLJ31688 fis, clone | I | 1.358247 | 0.001605 |
| | | NT2RI2005520 | | | |
| 203720_s_at | ERCC1 | excision repair cross- | I | 1.357248 | 0.003855 |
| | | complementing rodent repair | | | |
| | | deficiency, complementation | | | |
| | | group 1 (includes overlapping | | | |
| | | antisense sequence) | | | |
| 208829_at | TAPBP | TAP binding protein (tapasin) | I | 1.345765 | 0.00399 |
| 204986_s_at | TAOK2 | TAO kinase 2 | I | 1.344032 | 0.003593 |
| 1563920_at | FAM45A | family with sequence similarity | I | 1.339147 | 0.001689 |
| | | 45, member A | | | |
| 239035_at | MTHFR | 5,10- | I | 1.33693 | 0.001358 |
| | | methylenetetrahydrofolate | | | |
| | | reductase (NADPH) | | | |
| 213468_at | ERCC2 | excision repair cross- | I | 1.330536 | 0.001163 |
| | | complementing rodent repair | | | |
| | | deficiency, complementation | | | |
| | | group 2 (xeroderma | | | |
| | | pigmentosum D) | | | |
| 1562898_at | | MRNA; cDNA | I | 1.328182 | 0.001164 |
| | | DKFZp667K1916 (from clone | | | |
| | | DKFZp667K1916) | | | |
| 229296_at | | CDNA FLJ34873 fis, clone | I | 1.318305 | 0.004458 |
| | | NT2NE2014950 | | | |
| 222180_at | | CDNA FLJ14122 fis, clone | I | 1.30708 | 0.003782 |
| | | MAMMA1002033 | | | |
| 227103_s_at | ECE2 | endothelin converting enzyme 2 | I | 1.293211 | 0.004467 |
| 229784_at | MGC16121 | Hypothetical protein | I | 1.292052 | 0.004778 |
| | | MGC16121 | | | |
| 1569383_s_at | ZFYVE28 | zinc finger, FYVE domain | I | 1.288088 | 0.004953 |
| | | containing 28 | | | |
| 212686_at | PPM1H | protein phosphatase 1H | I | 1.288 | 0.00357 |
| | | (PP2C domain containing) | | | |
| 225778_at | FUT1 | fucosyltransferase 1 | I | 1.279503 | 0.0037 |
| | | (galactoside 2-alpha-L- | | | |
| | | fucosyltransferase, H blood | | | |
| | | group) | | | |
| 219991_at | SLC2A9 | solute carrier family 2 | I | 1.27353 | 0.004082 |
| | | (facilitated glucose | | | |
| | | transporter), member 9 | | | |
| 218473_s_at | GLT25D1 | glycosyltransferase 25 domain | I | 1.266737 | 0.002982 |
| | | containing 1 | | | |
| 1563708_at | SFXN5 | sideroflexin 5 | I | 1.26665 | 0.004316 |
| 207292_s_at | MAPK7 | mitogen-activated protein | I | 1.254476 | 0.003188 |
| | | kinase 7 | | | |
| 226060_at | RFT1 | RFT1 homolog (S. cerevisiae) | I | 1.253227 | 0.003322 |
| 238696_at | RP11- | Heterogeneous nuclear | D | 0.821197 | 0.002245 |
| | 78J21.1 | ribonucleoprotein A1-like | | | |
| 209109_s_at | TSPAN6 | tetraspanin 6 | D | 0.819537 | 0.002324 |
| 215253_s_at | RCAN1 | regulator of calcineurin 1 | D | 0.818836 | 0.001155 |
| 1552409_a_at | ODF4 | outer dense fiber of sperm | D | 0.800931 | 0.003832 |
| | | tails 4 | | | |
| 223735_at | ARL6 | ADP-ribosylation factor-like 6 | D | 0.800452 | 0.001532 |
| 206557_at | ZNF702 | zinc finger protein 702 | D | 0.793049 | 0.004966 |
| 238867_at | TMEM182 | transmembrane protein 182 | D | 0.78403 | 0.002723 |
| 227568_at | HECTD2 | HECT domain containing 2 | D | 0.776805 | 0.003742 |
| 1556694_a_at | | CDNA FLJ37138 fis, clone | D | 0.770416 | 0.002305 |
| | | BRACE2023718 | | | |
| 222097_at | | Transcribed locus | D | 0.770036 | 0.001371 |
| 234620_at | LOC402643 | tropomyosin 3 pseudogene | D | 0.750701 | 0.004015 |
| 226309_at | DNAL1 | dynein, axonemal, light chain 1 | D | 0.749849 | 0.001325 |
| 227502_at | KIAA1147 | KIAA1147 | D | 0.745079 | 0.003706 |
| 1553351_at | OTUD7A | OTU domain containing 7A | D | 0.73819 | 0.001381 |
| 223079_s_at | GLS | glutaminase | D | 0.731159 | 0.004769 |
| 239072_at | LOC647121 | embigin homolog (mouse) | D | 0.730558 | 0.001722 |
| | | pseudogene | | | |
| 1557098_s_at | HAR1A | highly accelerated region 1A | D | 0.72157 | 0.002867 |
| | | (non-protein-coding RNA) | | | |
| 236635_at | ZNF667 | zinc finger protein 667 | D | 0.71711 | 0.004602 |
| 229363_at | | CDNA FLJ32121 fis, clone | D | 0.691577 | 0.002885 |
| | | PEBLM1000083 | | | |
| 208650_s_at | CD24 | CD24 molecule | D | 0.552158 | 0.001825 |

I—increased in high delusions states(biomarker for high delusions);
D—decreased in high delusions state/increased in no delusions state (biomarker for no delusions).

1. A method of diagnosing psychosis in an individual, the method comprising
determining the expression of a plurality of biomarkers for delusion or hallucination in a sample from the individual, the plurality of biomarkers selected from the group of biomarkers listed in Table 5A, Table 5B, Table 6A, and Table 6B.

2. The method of claim 1, wherein the plurality of biomarkers comprise a subset of about 10 biomarkers for delusions designated as Drd2, ApoE, Scamp1, Idh1, Nab1, Nrg1, Egr1, Dctn1, Pllp, and Pvalb.

3. The method of claim 1, wherein the plurality of biomarkers comprise a subset of about 10 biomarkers for hallucinations designated as Rhobtb3, Aldh111, Mpp3, Fn1, Spp1, Arhgef9, S100a6, Adamts5, Pdap1, and Plxnd1.

4. The method of claim 1, wherein the sample is blood.

5. The method of claim 1, wherein the level of the biomarker is determined in a tissue biopsy sample of the individual.

6. The method of claim 1, wherein the level of the biomarker is determined by a method selected from the group consisting of analyzing the expression level of RNA transcripts, analyzing the level of protein, and analyzing the level of peptides or fragments thereof.

7. The method of claim 1, wherein the expression level is determined by an analytical technique selected from the group consisting of microarray gene expression analysis, polymerase chain reaction (PCR), real-time PCR, quantitative PCR, immunohistochemistry, enzyme-linked immunosorbent assays (ELISA), and antibody arrays.

8. The method of claim 1, wherein the determination of the level of the plurality of biomarkers is performed by an analysis for the presence or absence of the biomarkers.

9. A The method of claim 1 further comprising:
(a) assigning a predictive value or score to the level of the biomarkers; and
(b) diagnosing the psychosis based on the assigned value or score.

10. A method of predicting the probable course and outcome (prognosis) of psychosis, the method comprising:
(a) analyzing a test sample for the expression of a plurality of biomarkers of psychosis, the markers selected from the group consisting of biomarkers listed in Tables 3A and 3B; and
(b) determining the prognosis of the subject based on the expression of the biomarkers and one or more clinicopathological data to implement a particular treatment plan for the subject.

11. The method of claim 10, wherein the treatment plan is for delusion based on the expression of the biomarkers for delusion selected from the group consisting of Drd2, ApoE, Scamp1, Idh1, Nab1, Nrg1, Egr1, Dctn1, Pllp, and Pvalb.

12. The method of claim 10, wherein the treatment plan is for hallucination based on the expression of the biomarkers for delusion selected from the group consisting of Rhobtb3, Aldh111, Mpp3, Fn1, Spp1, Arhgef9, S100a6, Adamts5, Pdap1, and Plxnd1.

13. The method of claim 10, wherein the clinicopathological data is selected from the group consisting of patient age, previous personal and/or familial history of psychosis, previous personal and/or familial history of response to psychosis, and any genetic or biochemical predisposition to psychiatric illness.

14. The method of claim 10, wherein the test sample from the subject is of a test sample selected from the group consisting of fresh blood, stored blood, fixed, paraffin-embedded tissue, tissue biopsy, tissue microarray, fine needle aspirates, peritoneal fluid, ductal lavage and pleural fluid or a derivative thereof.

15. (canceled)

16. (canceled)

17. The method of claim 10, further comprising a personalized plan.

18. A diagnostic array for psychosis to detect the expression of a plurality of genes selected from the group of genes listed in Tables 5A-5B and 6A-6B.

19. The diagnostic array of claim 18 consisting essentially of biomarkers listed in Table 3A-3B.

20. The diagnostic array of claim 19 consisting essentially of biomarkers designated as Drd2, ApoE, Scamp1, Idh1, Nab1, Nrg1, Egr1, Dctn1, Pllp, and Pvalb for delusion and Rhobtb3, Aldh111, Mpp3, Fn1, Spp1, Arhgef9, S100a6, Adamts5, Pdap1, and Plxnd1 for hallucination.

21. (canceled)

22. The diagnostic array of claim 18 that detects the protein levels of the biomarkers from a blood sample.

23. (canceled)
